(19) United States
(12) Patent Application Publication
(10) Pub. No.: US 2009/0076199 A1
(43) Pub. Date: Mar. 19, 2009

(54) RED DYE MIXTURE

(75) Inventors: Stephan Michaelis, Odenthal (DE); Horst Berneth, Leverkusen (DE); Eberhard Kuckert, Leverkusen (DE)

Correspondence Address: LANXESS CORPORATION, 111 RIDC PARK WEST DRIVE, PITTSBURGH, PA, 15275-1112, US

(73) Assignee: LANXESS DEUTSCHLAND GMBH, Leverkusen (DE)

(21) Appl. No.: 12/208,418

(22) Filed: Sep. 11, 2008

(30) Foreign Application Priority Data
Sep. 18, 2007 (DE) 102007044743.6
May 9, 2008 (DE) 102008023114.2

(51) Int. Cl. C08K 5/3417; C08K 5/3432; C08K 5/3437; C07C 211/61

(52) U.S. Cl. 524/90; 524/99; 524/94; 524/87; 564/315; 564/427

(57) ABSTRACT

Mixtures have been found comprising at least one red dye of the formula (I), (C) or (CI)

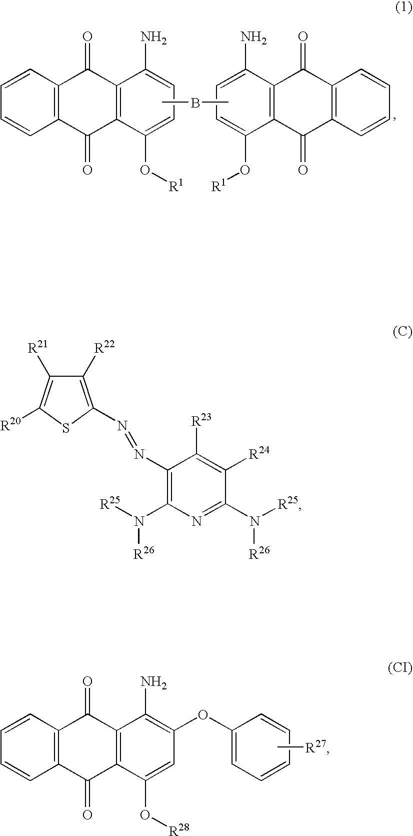

and at least one orange dye of the formula (L), (LI), (LII), (LIII), (LIV) or (LV)

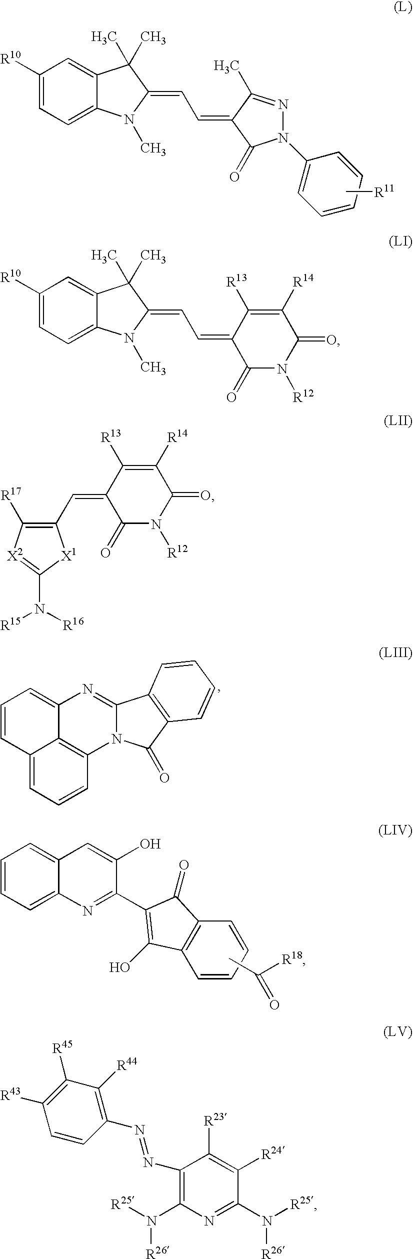

where the substituents have the definition in the description, these mixtures being suitable more particularly for the mass colouring of plastics.

The invention relates to mixtures comprising specific red dyes and specific orange dyes, to masterbatches based thereon and to their use for the mass colouring of plastics, to the coloured plastics themselves, and also to new specific red dyes and processes for preparing them.

Recent years have seen a sharp rise in the demand for brown plastic bottles, more particularly PET bottles. Colouring to date has used a brown dye mixture comprising as its red dye C.I. Solvent Red 135, the perinone of the formula

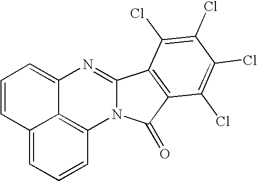

and also a further yellow dye and green dye. This colour mixture has established a very widespread brown hue. The coloristic mandate of this brown hue, on the one hand, is opposed on the other hand by heightened performance requirements and also by the need for chlorine-free substitutes. As well as high lightfastness, thermal stability and colour strength, and high solubility in the plastic, sublimation fastness and migration fastness qualities are also demanded.

It was an object of the present invention, therefore, to find a coloristic substitute for the red component of the brown mixture which at the same exceeds the required performance properties.

A mixture has now been found comprising at least one red dye of the formula (I), (C) or (CI)

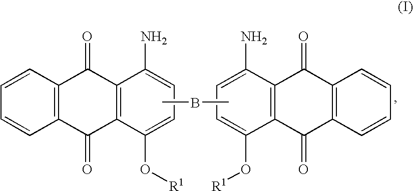

more particularly of the formula (Ia)

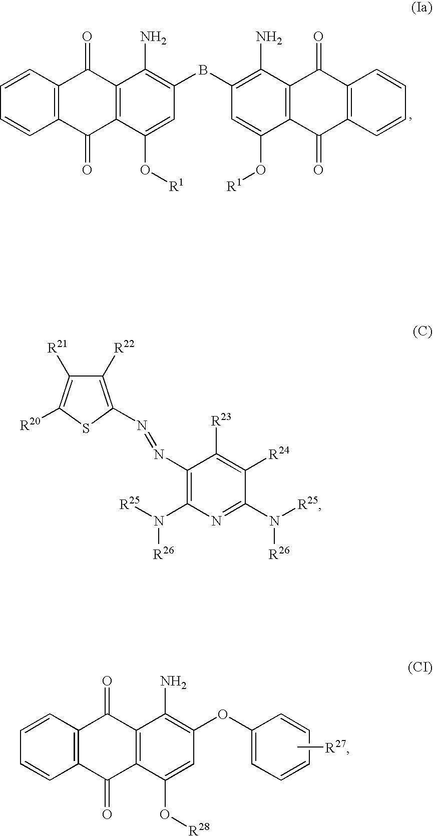

in which
R¹ is hydrogen, optionally substituted C₁-C₈-alkyl or optionally substituted C₃-C₈-cycloalkyl,
B is a bridge of the formulae —O—B¹—O— or —CH₂—B²—CH₂—,
B¹ is a bridge of the formula

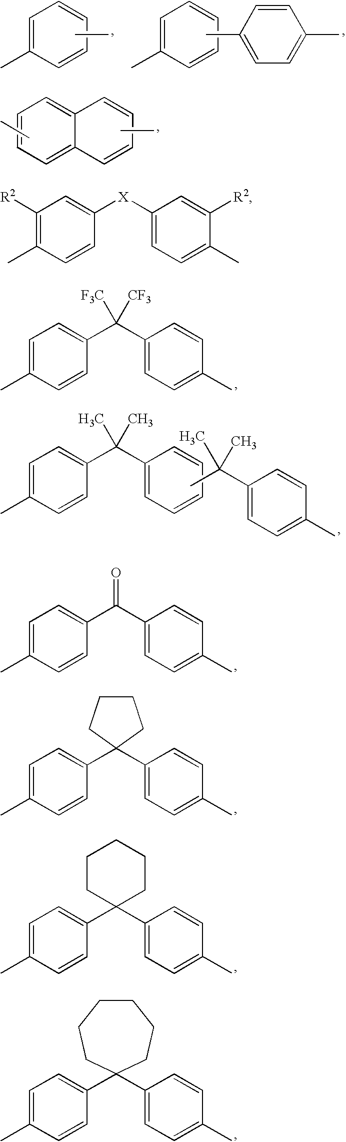

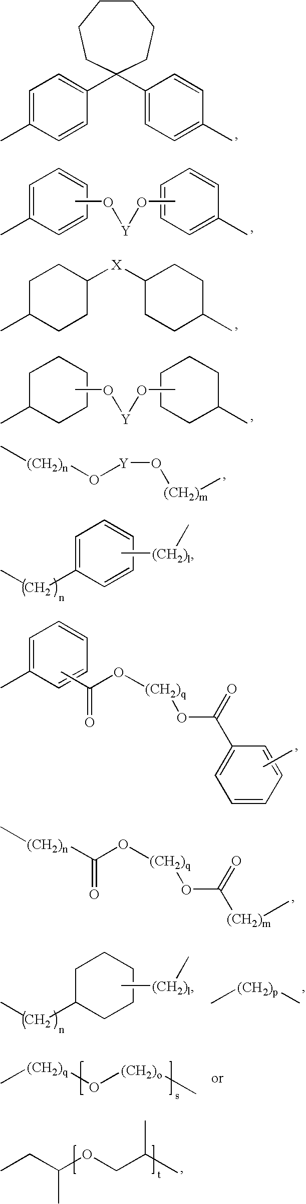

B² is a bridge of the formula

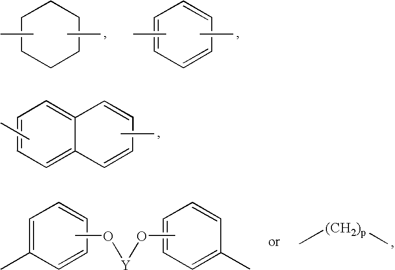

R² is hydrogen, methyl, ethyl, methoxy or fluorine,
X is a bridge of the formula

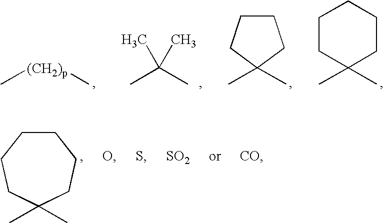

Y is a bridge of the formula

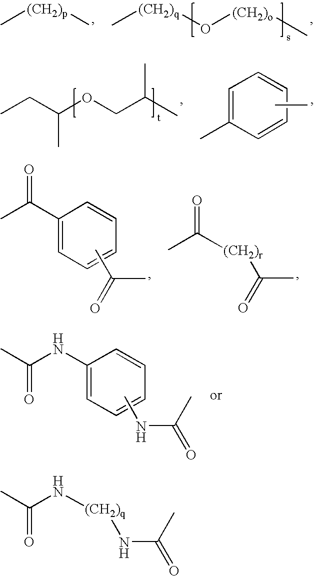

l is an integer from 0 to 8,
m, n, p and r independently of one another are an integer from 1 to 8,
q is an integer from 2 to 8 and
s and t independently of one another are an integer from 1 to 4,
and
R²⁰ and R²² independently of one another are cyano or optionally substituted C₁-C₈-alkoxycarbonyl,
R²¹ is hydrogen or optionally substituted C₁-C₃-alkyl,
R²³ is hydrogen, optionally substituted C₁-C₈-alkyl or optionally substituted C₁-C₈-alkoxycarbonyl,
R²⁴ is cyano, optionally substituted C₁-C₈-alkylsulphonyl or optionally substituted C₁-C₈-alkoxycarbonyl,
R²⁵ is optionally substituted C₁-C₈-alkyl, optionally substituted C₃-C₇-cycloalkyl, optionally substituted C₆-C₁₀-aryl,
R²⁶ is hydrogen or independently of R²⁵ has the definition of R²⁵, or
NR²⁵R²⁶ is pyrrolidino, piperidino or morpholino,
R²⁷ is one or more radicals having the definition hydrogen, optionally substituted C₁-C₈-alkyl, optionally substituted C₁-C₈-alkoxy, halogen, cyano, optionally substituted C₃-C₇-cycloalkyl or optionally substituted C₆-C₁₀-aryl,
R²⁸ is hydrogen or optionally substituted C₁-C₈-alkyl,
and at least one orange dye of the formula
(L), (LI), (LII), (LIII), (LIV) or (LV)

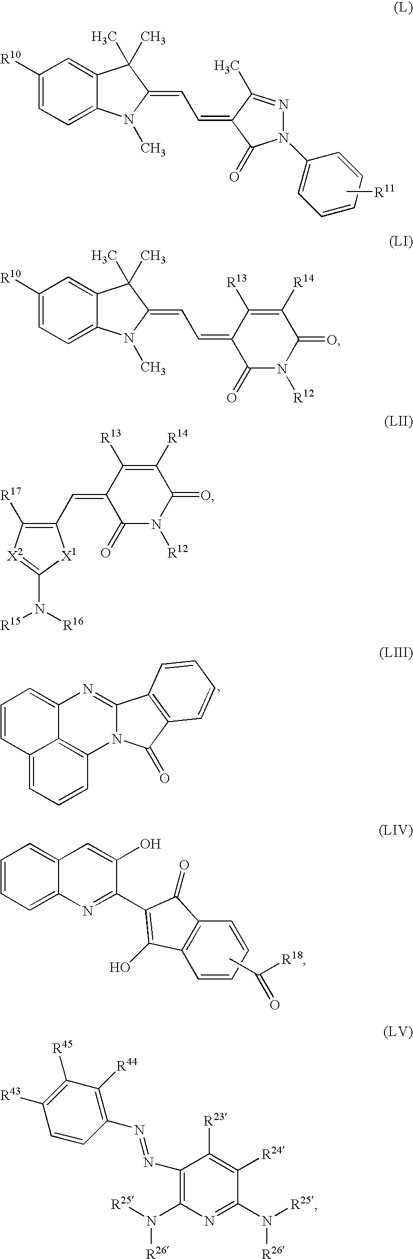

in which
R¹⁰ is hydrogen, optionally substituted C₁-C₈-alkyl, optionally substituted C₁-C₈-alkoxy, optionally substituted C₁-C₈-alkylsulphonyl, optionally substituted C₁-C₈-alkoxycarbonyl, nitro, cyano or fluorine,
R¹¹ is hydrogen, optionally substituted C₁-C₈-alkyl, optionally substituted C₁-C₈-alkoxy, optionally substituted C₁-C₈-alkoxycarbonyl, nitro, cyano or fluorine,
R¹² is hydrogen, optionally substituted C₁-C₈-alkyl or optionally substituted C₃-C₈-cycloalkyl,
R¹³ is hydrogen, optionally substituted C₁-C₈-alkyl or optionally substituted C₁-C₈-alkoxycarbonyl,
R¹⁴ is cyano, optionally substituted C₁-C₈-alkylsulphonyl or optionally substituted C₁-C₈-alkoxycarbonyl,
X¹ is O or S,
X² is N or C—CN,
R¹⁵ and R¹⁶ independently of one another are optionally substituted C₁-C₈-alkyl, optionally substituted C₃-C₈-cycloalkyl or optionally substituted C₆-C₁₀-aryl, or
NR¹⁵R¹⁶ is pyrrolidino, piperidino or morpholino,
R¹⁷ is hydrogen or C₁-C₈-alkyl,
R¹⁸ is optionally substituted C₆-C₁₀-aryl,
R²³′ is hydrogen, optionally substituted C₁-C₈-alkyl or optionally substituted C₁-C₈-alkoxycarbonyl,
R²⁴′ is cyano, optionally substituted C₁-C₈-alkylsulphonyl or optionally substituted C₁-C₈-alkoxycarbonyl,
R²⁵′ is optionally substituted C₁-C₈-alkyl, optionally substituted C₃-C₇-cycloalkyl, optionally substituted C₆-C₁₀-aryl,
R²⁶′ is hydrogen or independently of R²⁵′ has the definition of R²⁵′, or
NR²⁵′R²⁶′ is pyrrolidino, piperidino or morpholino,
R⁴³ and R⁴⁴ independently of one another are hydrogen, optionally substituted C₁-C₄-alkyl, more particularly mono- or poly-fluorine-substituted C₁-C₄-alkyl, nitro, cyano, C₁-C₄-alkoxycarbonyl, and
R⁴⁵ is hydrogen, methyl, cyano, fluorine or chlorine.

The compound of the formula I preferably corresponds to the formulae (Ia) to (Ic)

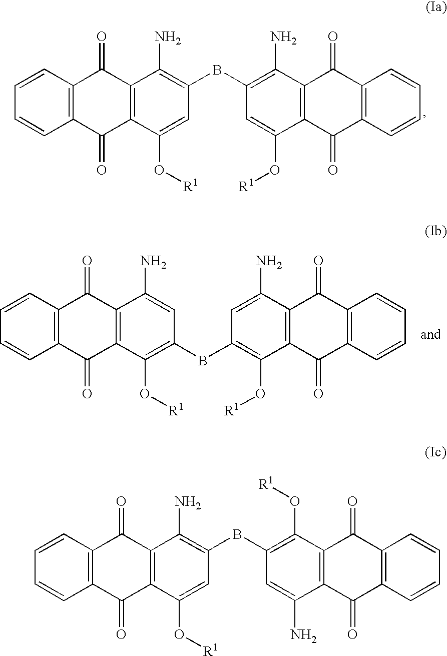

more particularly of the formula Ia.

In the case of the naphthalene structure the undefined bonds attach preferably in positions 1,5-, 1,8-, 2,6- or 2,7-. In the case of the benzene and cyclohexane structures the undefined bonds attach in positions 1,2-, 1,3- or 1,4-.

By C₁-C₈-alkyl here and below is meant linear and/or branched C₁-C₈-alkyl, e.g. methyl, ethyl, n-propyl, 2-propyl, n-butyl, 2-butyl, isobutyl, tert-butyl, pentyl, hexyl, heptyl, octyl or 2-ethylhexyl.

By substituted C₁-C₈-alkyl is meant linear and/or branched C₁-C₈-alkyl, as described above, which is substituted by at least one of the radicals fluorine, cyano, hydroxyl, C₁-C₄-alkoxy, acetoxy, propoxy, butoxy, C₁-C₄-alkoxycarbonyl, cyclopentyl, cyclohexyl, phenyl, naphthyl e.g. trifluoromethyl, perfluorobutyl, 2-chloroethyl, cyanomethyl, 2-cyanoethyl, 2-methoxyethyl, 2-ethoxyethyl, 2-acetoxyethyl, 2-methoxycarbonylethyl, cyclohexylmethyl, benzyl, phenethyl or phenylpropyl.

Here and below, for example, butyl is always n-butyl, 2-butyl, isobutyl or tert-butyl, unless expressly stated otherwise.

By C₃-C₈-cycloalkyl is meant, here and below, cyclopropyl, cyclobutyl, cyclopentyl, cyclohexyl, cycloheptyl or cyclooctyl. By substituted C₃-C₈-cycloalkyl is meant, for example, methylcyclohexyl.

Preferably R¹ is hydrogen. Likewise preferably R¹ is methyl.

Preferred compounds of the formula I are those in which
B¹ is a bridge of the formula

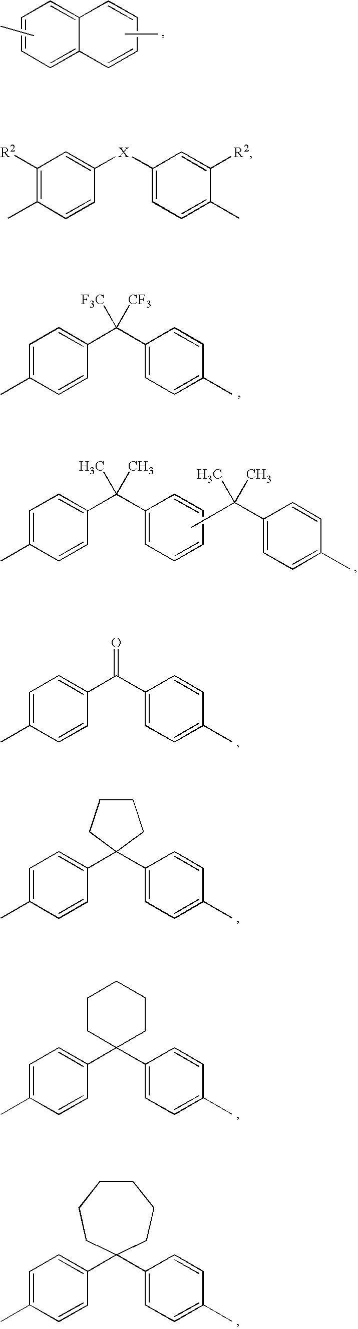

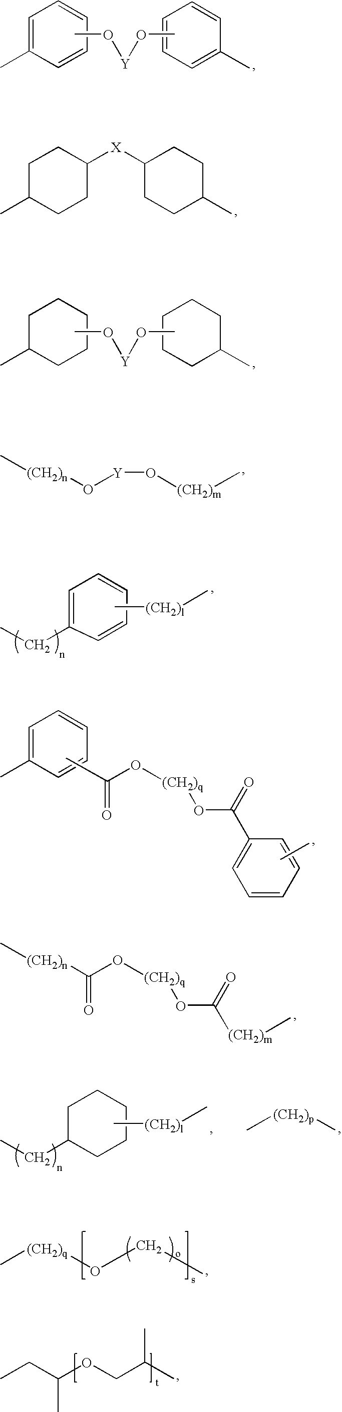

R² is methyl, ethyl, methoxy or fluorine,
X is a bridge of the formulae

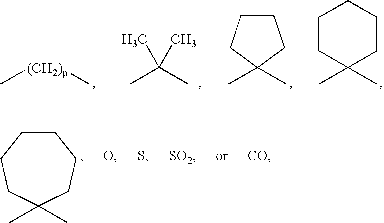

Y is a bridge of the formulae

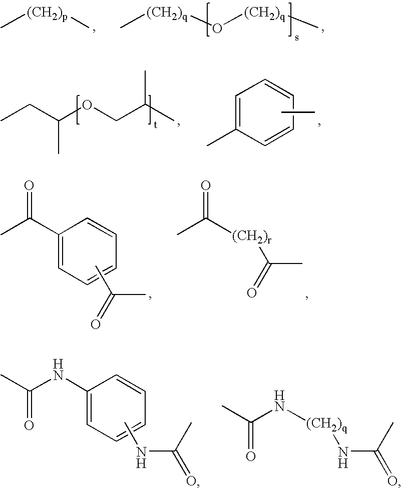

l is an integer from 0 to 8,
m, n, p and r independently of one another are an integer from 1 to 8,
q is an integer from 2 to 8 and
s and t independently of one another are an integer from 1 to 4.

Particularly preferred bridges of the formula B¹ are those

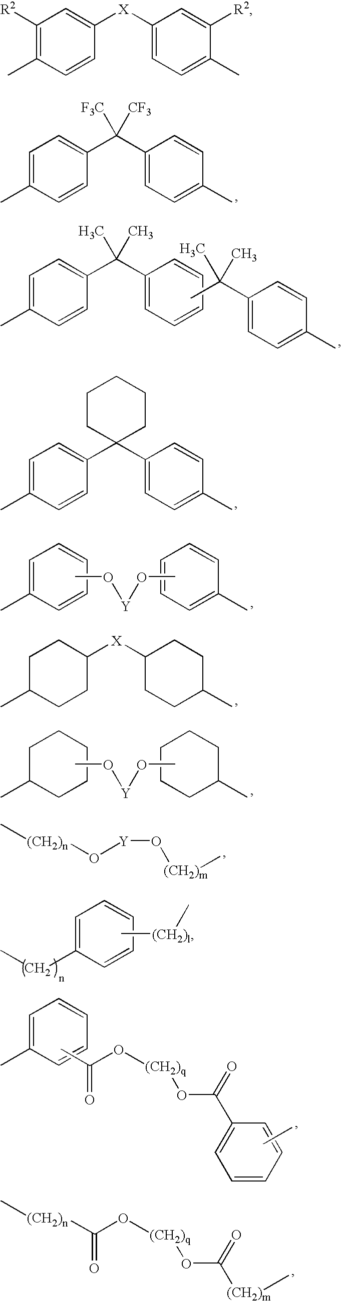

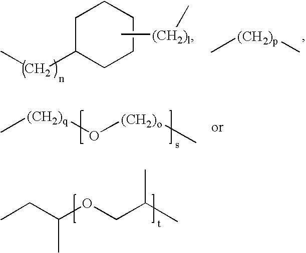

in which
R² is methyl,
X is a bridge of the formula

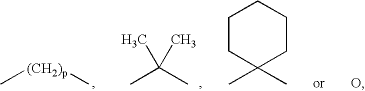

Y is a bridge of the formula

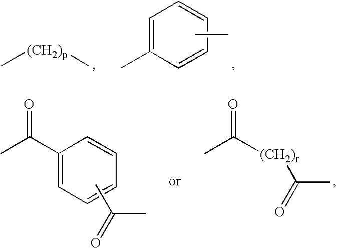

l is an integer from 0 to 4,
m, n and p independently of one another are an integer from 1 to 4,
q and r independently of one another are an integer from 2 to 6,
s and t independently of one another are an integer from 1 to 2 and
the undefined ring positions are 1,2-, 1,3- or 1,4-.

Especially preferred bridges B¹ are:

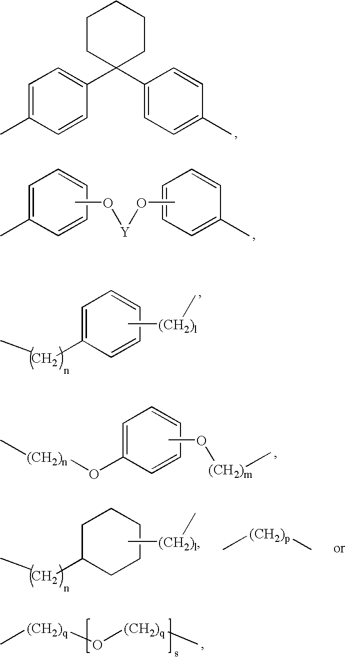

in which
Y is a bridge of the formula

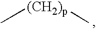

l is an integer from 1 to 2,
m, n and p independently of one another are an integer from 1 to 2,
q is an integer from 2 to 4,
s and t independently of one another are 1 and
the undefined ring positions are 1,4-.

Preferred bridges B² are:

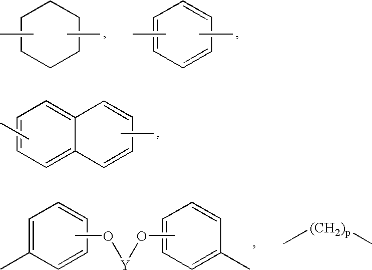

in which
Y is a bridge of the formulae

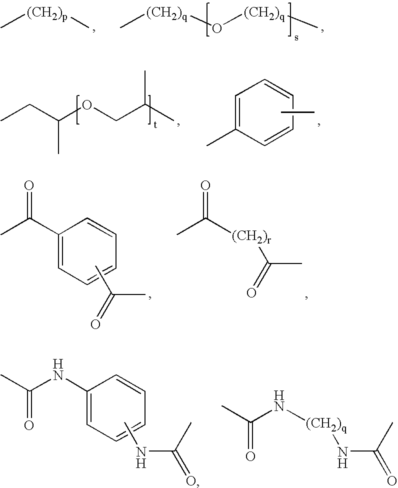

p and r independently of one another are an integer from 1 to 8,
q is an integer from 2 to 8 and
s and t independently of one another are an integer from 1 to 4.

Particularly preferred bridges B² are

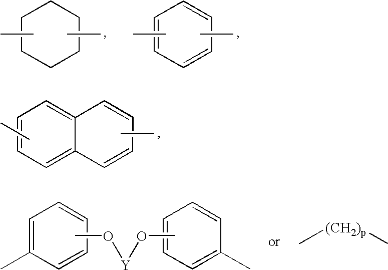

in which
Y is a bridge of the formula

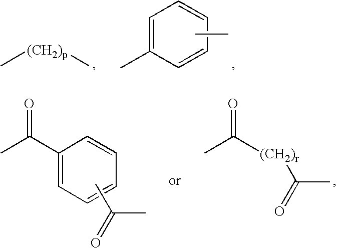

p is an integer from 1 to 6 and
r is an integer from 2 to 6
   and the undefined ring positions in the case of benzene or cyclohexane rings are 1,2-, 1,3- or 1,4- and in the case of the napththalene ring are 2,6- or 2,7-.

Especially preferred bridges B² are:

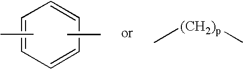

in which
p is an integer from 1 to 4
   and the undefined ring positions are 1,4-.

Preferred red dyes of the formula (C) are those in which
R²⁰ is cyano, methoxycarbonyl or ethoxycarbonyl, more preferably cyano or methoxycarbonyl, very preferably methoxycarbonyl,
R²² is cyano,
R²¹ is hydrogen or methyl, more preferably methyl,
R²³ is methyl, trifluoromethyl or methoxycarbonyl, more preferably methyl,
R²⁴ für cyano, methanesulphonyl, methoxycarbonyl or ethoxycarbonyl, more preferably cyano,
R²⁵ is methyl, ethyl, propyl, butyl, methoxyethyl, methoxypropyl, cyanoethyl, benzyl, cyclohexyl, phenyl or tolyl, more preferably ethyl, propyl, butyl, methoxyethyl, methoxypropyl, benzyl, cyclohexyl or phenyl, very preferably propyl, butyl or methoxypropyl,
R²⁶ is hydrogen or independently of R²⁵ has the definition of R²⁵, or
NR²⁵R²⁶ is pyrrolidino, piperidino or morpholino.

Preferred red dyes of the formula (CI) are those in which
R²⁷ is hydrogen, methyl, ethyl, propyl, butyl, benzyl, cyano, cyclohexyl, phenyl or tolyl, more preferably p-positioned methyl, ethyl, propyl, butyl, cyclohexyl or phenyl, very preferably p-positioned butyl or cyclohexyl, and
R²⁸ is hydrogen.

The dyes of the formula (I) and at least one orange dye of the formula (L) to (LV) are mixed preferably in a ratio of 1:99 to 99:1, preferably of 20:80 to 97:3, more preferably of 40:60 to 95:5, very preferably 50:50 to 90:10.

The red dyes of the formula (C) and at least one orange dye of the formula (L) to (LV) are mixed preferably in a ratio at 1:99 to 99:1, preferentially 20:80 to 97:3, more preferably 30:70 to 95:5, very preferably from 40:60 to 90:10. Very particular preference is given to a mixture of the dyes of the formula (C) and (L) in a ratio of 40:60 to 60:40.

The dyes of the formula (CI) and at least one orange dye of the formula (L) to (LV) are mixed preferably in a ratio of 1:99 to 99:1, preferably of 20:80 to 97:3, more preferably of 40:60 to 95:5, very preferably 50:50 to 90:10.

Mixing may take place, for example, by the mixing of the dyes in solid form, in the form for example of powders, granules or pastes. In the case of wet pastes, for example, drying may follow. Mixing may also take place by mixing suspensions of the dyes, which subsequently for example are spray-dried or spray-granulated. Mixing, finally, may also take place during the production of masterbatches or during the colouring of polymeric materials.

Suitable colour loci for red mixtures of the invention are, according to the Lab values, L of 35-60, a of 65-85 and b of 20-75, preferably L of 40-50, a of 65-75 and b of 40-75.

For the orange dyes (L) to (LV) preferably
R¹⁰ is hydrogen, methyl, ethyl, methoxy, methanesulphonyl, methoxycarbonyl, ethoxycarbonyl, nitro, cyano or fluorine, more preferably hydrogen, methanesulphonyl or methoxycarbonyl, very preferably methoxycarbonyl,
R¹¹ is hydrogen, methyl, methoxy, methoxycarbonyl, nitro, cyano or fluorine, more preferably hydrogen,
R¹² is methyl, ethyl, propyl, butyl, 2-methoxyethyl, 2-cyanoethyl, benzyl or cyclohexyl, more preferably methyl, ethyl, propyl, butyl or benzyl, very preferably methyl or ethyl,
R¹³ is methyl or methoxycarbonyl, more preferably methyl,
R¹⁴ is cyano, methanesulphonyl, methoxycarbonyl or ethoxycarbonyl, more preferably cyano,
X¹ is O or S, more preferably S,
X² is N or C—CN,
R¹⁵ and R¹⁶ independently of one another are methyl, ethyl, propyl, butyl, cyanoethyl, benzyl, cyclopentyl, cyclohexyl, phenyl or tolyl, more preferably methyl, ethyl, cyanoethyl, benzyl, cyclohexyl or phenyl, very preferably methyl or ethyl, or
NR¹⁵R¹⁶ is pyrrolidino, piperidino or morpholino,
R¹⁷ is hydrogen or methyl, more preferably hydrogen,
R¹⁸ is phenyl, tolyl, dimethylphenyl, trimethylphenyl, more preferably tolyl or dimethylphenyl,
R²³′ is methyl, trifluoromethyl or methoxycarbonyl,
R²⁴′ is cyano, methylsulphonyl, trifluoromethylsulphonyl, methoxy- or ethoxycarbonyl,
R²⁵′ is methyl, ethyl, propyl, butyl, chloroethyl, methoxyethyl, methoxypropyl, dimethylaminopropyl, phenyl, tolyl, methoxyphenyl, fluorophenyl, chlorophenyl or cyanophenyl,
R²⁶′ is hydrogen or
   NR²⁵′R²⁶′ is pyrrolidino, piperidino or morpholino,
R⁴³ and R⁴⁴ independently of one another are trifluoromethyl, nitro, cyano, methoxy- or ethoxycarbonyl, it being possible for one of the two additionally to be hydrogen,
R⁴⁵ is hydrogen.

One preferred inventive mixture comprises a red dye of the formula (I) and at least one orange dye of the formula (L), (LI), (LII), (LIII), (LIV) or (LV), more particularly of the formula (L).

One likewise preferred inventive mixture comprises a red dye of the formula (C) and at least one orange dye of the formula (L), (LI), (LII), (LIII), (LIV) or (LV), more particularly of the formula (L).

One likewise preferred inventive mixture comprises a red dye of the formula (CI) and at least one orange dye of the formula (L), (LI), (LII), (LIII), (LIV) or (LV), more particularly of the formula (L).

Particular preference is given to an inventive mixture comprising a red dye of the formula (C), in which
R²⁰ is cyano, methoxycarbonyl or ethoxycarbonyl, more preferably cyano or methoxycarbonyl, very preferably methoxycarbonyl,
R²² is cyano,
R²¹ is hydrogen or methyl, more preferably methyl,
R²³ is methyl or methoxycarbonyl, more preferably methyl,
R²⁴ is cyano, methanesulphonyl, methoxycarbonyl or ethoxycarbonyl, more preferably cyano,
R²⁵ is methyl, ethyl, propyl, butyl, methoxyethyl, methoxypropyl, cyanoethyl, benzyl, cyclohexyl, phenyl or tolyl, more preferably ethyl, propyl, butyl, methoxyethyl, methoxypropyl, benzyl, cyclohexyl or phenyl, very preferably propyl, butyl or methoxypropyl,
R²⁶ is hydrogen or independently of R²⁵ has the definition of R²⁵, or
NR²⁵R²⁶ is pyrrolidino, piperidino or morpholino,
   more particularly when
R²⁰ is COOCH₃, R²¹ and R²³ are CH₃, R²² and R²⁴ are CN, R²⁵ is —CH₂CH₂CH₂OCH₃ and R²⁶ is H
   and an orange dye of the formula (L), more particularly in which
R¹⁰ is COOCH₃ and R¹¹ is H.

Examples of such mixtures are:

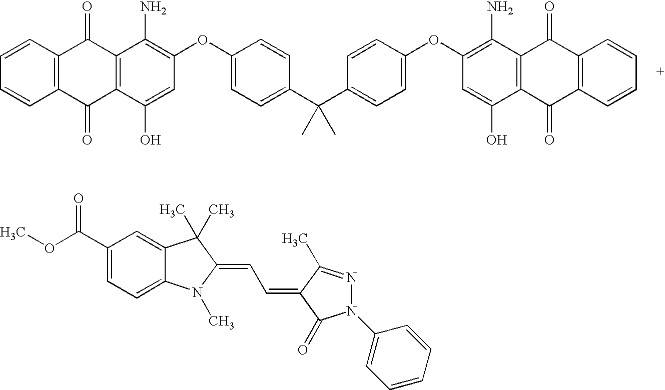

preferably in a ratio of 50:50,

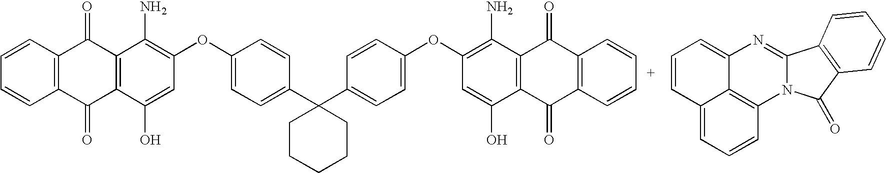

preferably in a ratio of 45:55,

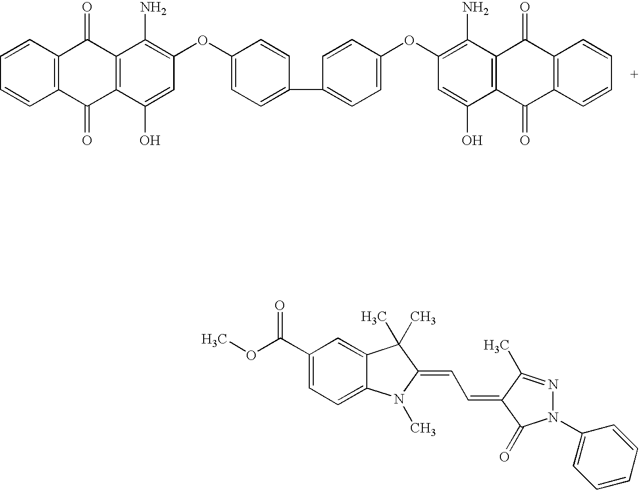

preferably in a ratio of 83:17,

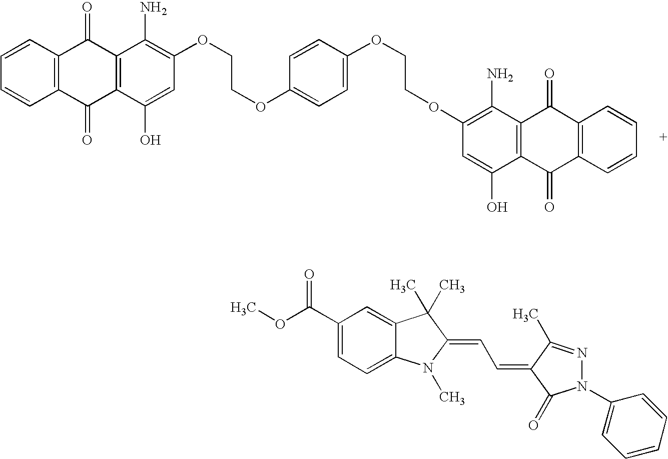

preferably in a ratio of 83:17,

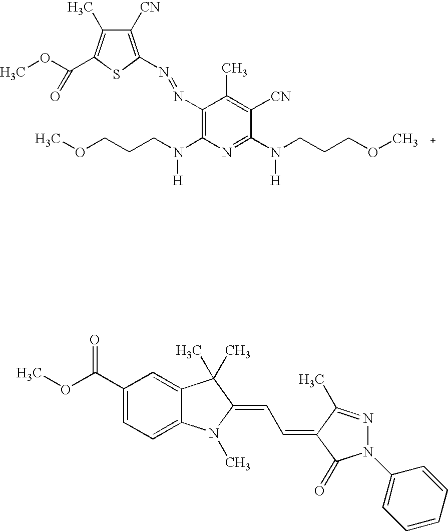

preferably in a ratio of 50.8:48.2,

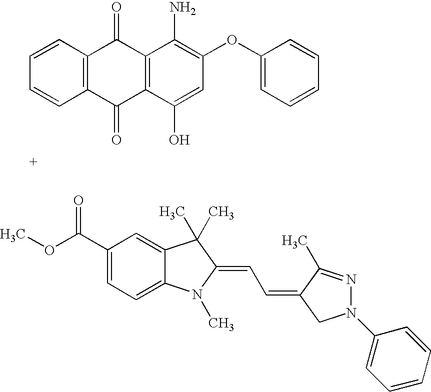

preferably in a ratio of 48:52,

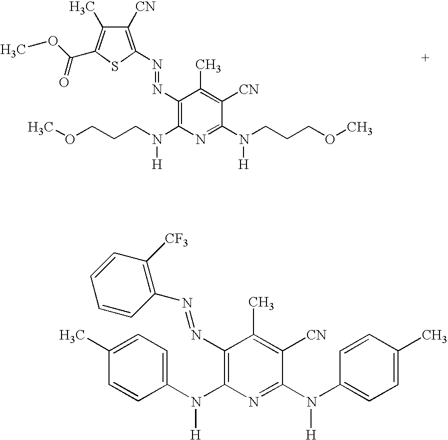

preferably in a ratio of 50:50.

The mixtures of the invention, where their only chromophoric components are red dyes and orange dyes, may be used for the mass colouring of plastics together with further dyes, more particularly those for obtaining a brown hue, or else the further dyes may already be present in the mixture.

The mixtures of the invention therefore preferably comprise
a) at least one yellow dye, preferably one with a λₘₐₓ of 420 to 460 nm and at least one green dye, preferably with a λₘₐₓ of 610 to 700 nm, or
b) at least one yellow dye, preferably one with a λₘₐₓ of 420 to 460 mm and at least one blue dye, preferably one with a λₘₐₓ of 570 to 640 nm.

Suitability as particularly preferred yellow dyes is possessed, for example, by those of the formulae

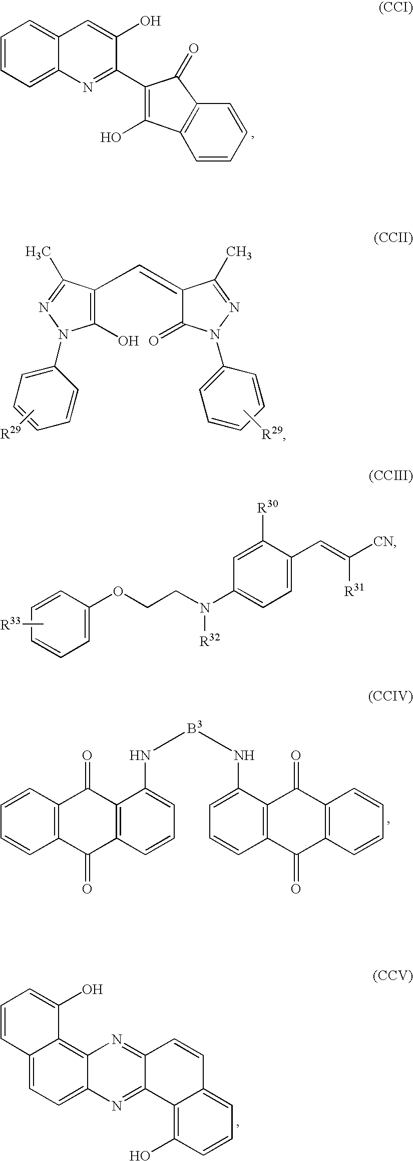

in which
R²⁹ is one or more radicals having the definition hydrogen, optionally substituted C₁-C₈-alkyl, optionally substituted C₁-C₈-alkoxy, fluorine, cyano, optionally substituted C₃-C₇-cycloalkyl or optionally substituted C₆-C₁₀-aryl,
R³⁰ is hydrogen, methyl, ethyl or methoxy,
R³¹ is cyano, C₁-C₈-alkoxycarbonyl or C₁-C₈-alkanesulphonyl,
R³² is hydrogen, optionally substituted C₁-C₈-alkyl or optionally substituted C₃-C₇-cycloalkyl,
R³³ is hydrogen, optionally substituted C₁-C₈-alkyl, optionally substituted C₁-C₈-alkoxy, optionally substituted C₃-C₇-cycloalkyl or optionally substituted C₆-C₁₀-aryl,
B³ is a bridge of the formula

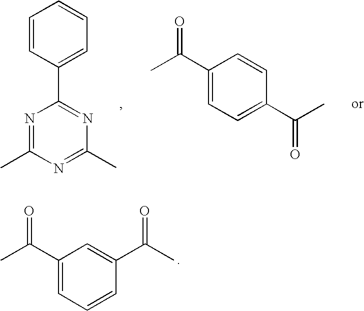

Preferably
R²⁹ is hydrogen, methyl, ethyl, propyl, butyl, benzyl, cyano, cyclohexyl, phenyl or tolyl, more preferably p-positioned methyl, ethyl, propyl, butyl, cyclohexyl or phenyl, very preferably p-positioned butyl or cyclohexyl,
R³⁰ is hydrogen or methyl,
R³¹ is cyano, methoxycarbonyl, ethoxycarbonyl or methanesulphonyl, more preferably cyano or methoxycarbonyl, very preferably cyano,
R³² is methyl, ethyl, propyl or cyclohexyl, more preferably methyl or ethyl,
R³³ is methyl, ethyl, propyl, butyl, pentyl, hexyl, heptyl, octyl, methoxy, ethoxy, cyclopentyl, cyclohexyl, phenyl or tolyl, more preferably p-positioned butyl, pentyl, hexyl, heptyl, octyl, cyclohexyl or phenyl, very preferably p-positioned n-butyl, tert-butyl, 1,1,3,3-tetramethylbutyl, 2-ethylhexyl, cyclohexyl or phenyl,
   and
B³ is a bridge of the formula

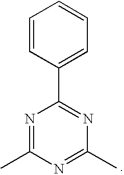

Suitability as particularly preferred green dyes is possessed, for example, by those of the formula (CCLI)

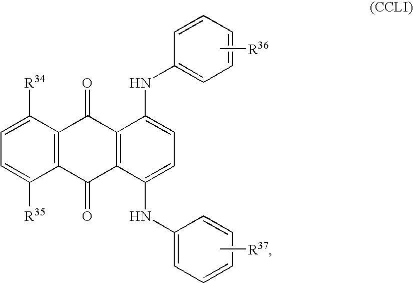

in which
R³⁴ and R³⁵ independently of one another are hydrogen, hydroxyl or C₁-C₄-alkoxy,
R³⁶ and R³⁷ independently of one another are hydrogen, optionally substituted C₁-C₈-alkyl, optionally substituted C₁-C₈-alkoxy, fluorine, optionally substituted C₃-C₇-cycloalkyl or optionally substituted C₆-C₁₀-aryl.

Preferably
R³⁴ and R³⁵ are hydrogen or hydroxyl, more preferably hydrogen, and are alike,
R³⁶ and R³⁷ independently of one another are hydrogen, methyl, ethyl, propyl, butyl, methoxy, bromine, cyclohexyl or phenyl, more preferably p-positioned methyl, ethyl, butyl, very preferably both equally p-positioned methyl or tert-butyl.

Suitability as particularly preferred blue dyes is possessed by those of the formula (CCCI)

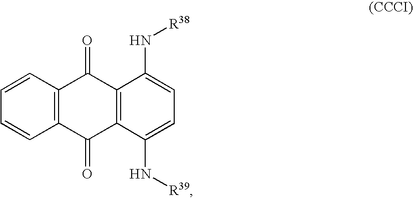

in which
R³⁸ and R³⁹ independently of one another are optionally substituted C₁-C₈-alkyl, optionally substituted C₃-C₇-cycloalkyl or optionally substituted C₆-C₁₀-aryl.

Preferably
R³⁸ and R³⁹ independently of one another are methyl, ethyl, propyl, butyl, pentyl, hexyl, cyclopentyl, cyclohexyl or a phenyl radical of the formula

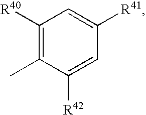

R⁴⁰ is methyl, ethyl or propyl,
R⁴¹ is hydrogen, methyl, ethyl, propyl, butyl or cyclohexyl,
R⁴² is hydrogen, methyl, ethyl or propyl.

More preferably
R³⁸ and R³⁹ are methyl, ethyl, propyl, butyl, cyclohexyl or a phenyl radical of the formula

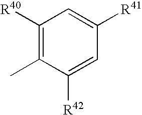

and are alike,
R⁴⁰ is methyl or ethyl,
R⁴¹ is hydrogen, methyl or ethyl,
R⁴² is methyl or ethyl.

The mixtures of the invention comprising at least one red dye of the formula (I) and at least one orange dye of the formula (L) to (LV) are further preferably mixed with a yellow dye and with a green or blue dye in a ratio of [(I)+(L) to (LV)]:yellow:[green and/or blue] of 30-70:20-50:1-30, preferably 45-65:25-40:15-25.

The mixtures of the invention comprising at least one red dye of the formula (C) and at least one orange dye of the formula (L) to (LV) are further preferably mixed with a yellow dye and with a green or blue dye in a ratio of [(C) and/or (CI)+(L) to (LV)]:yellow:[green and/or blue] of 15-40:20-70:20-50, preferably 20-35:25-65:25-40.

The mixtures of the invention comprising at least one red dye of the formula (CI) and at least one orange dye of the formula (L) to (LV) are further preferably mixed with a yellow dye and with a green or blue dye in a ratio of [(I)+(L) to (LV)]:yellow:[green and/or blue] of 30-70:20-50:1-30, preferably 45-65:25-40:15-25.

Suitable colour loci for such brown mixtures in the Lab system are, for example, L of 30-80, a of 10-30 and b of 40-80, preferably L of 50-75, a of 10-25 and b of 42-70.

Examples are:

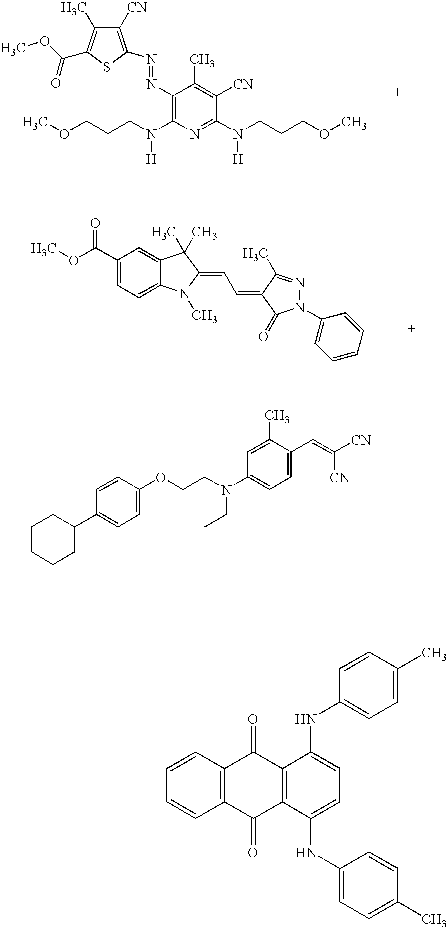

preferably in a ratio of 16.3:15.8:40.9:27.0,

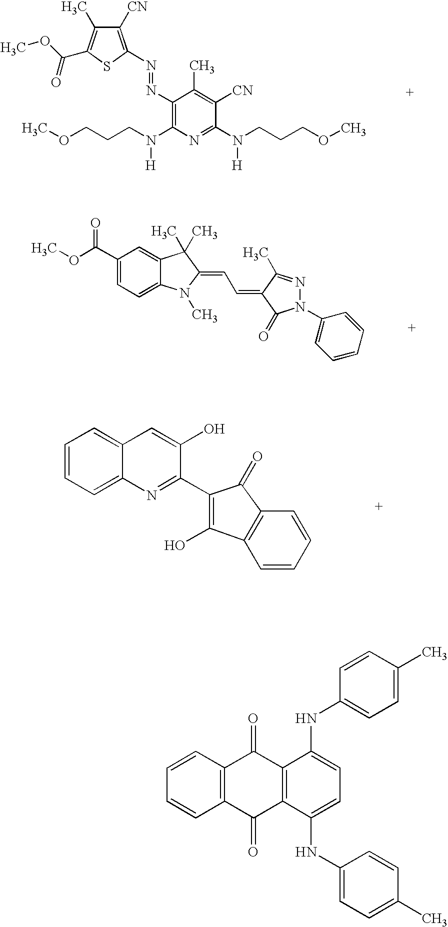

preferably in a ratio of 16.0:15.5:41.8:26.7,

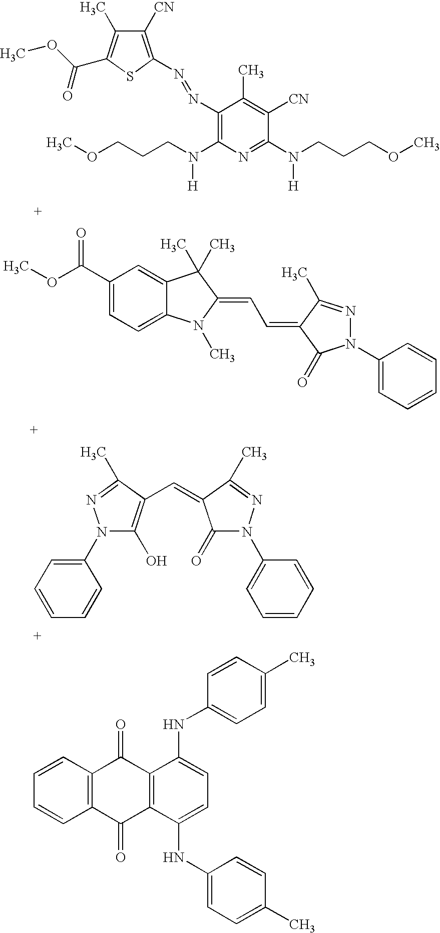

in a ratio of 9.0:8.7:66.7:15.6,

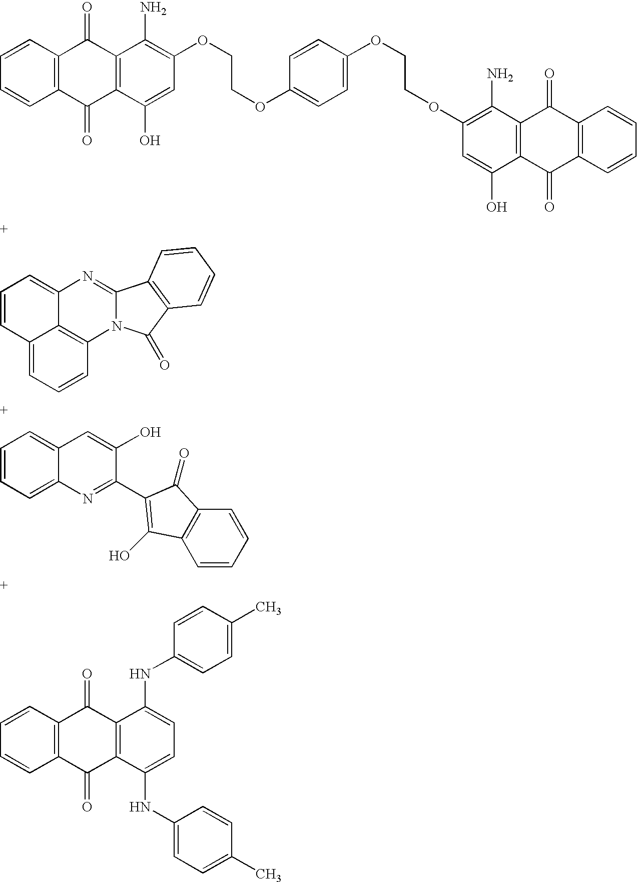

preferably in a ratio of 33.8:20.1:27.1:18.0,

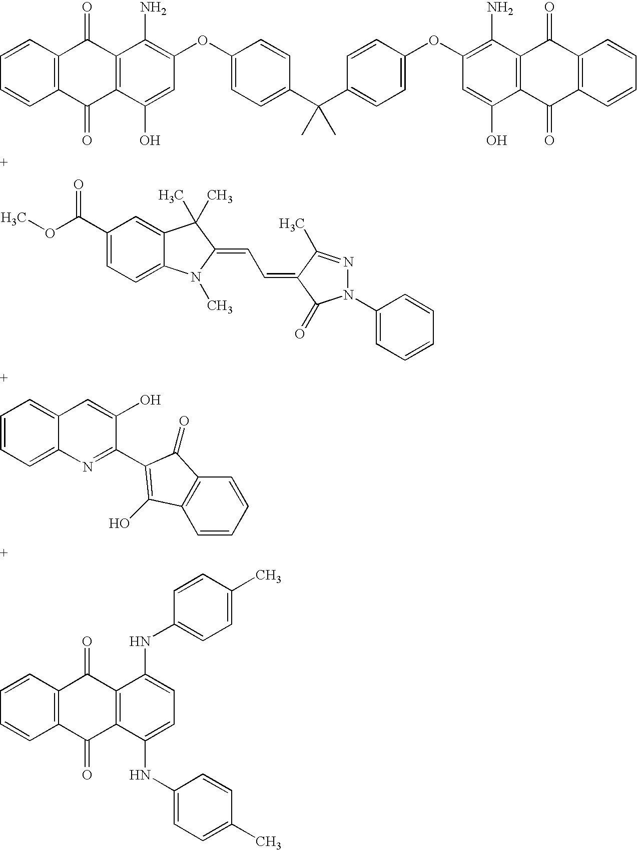

preferably in a ratio of 32.1:19.4:29.1:19.4,

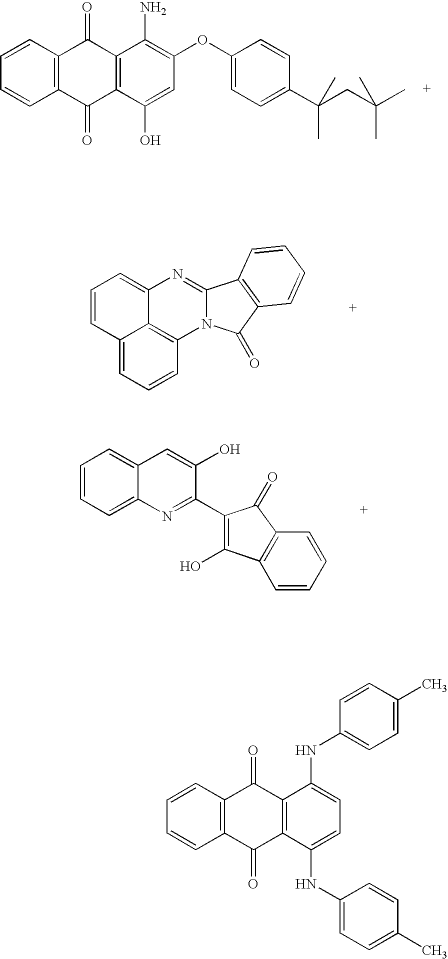

preferably in a ratio of 38.1:17.3:26.8:17.8,

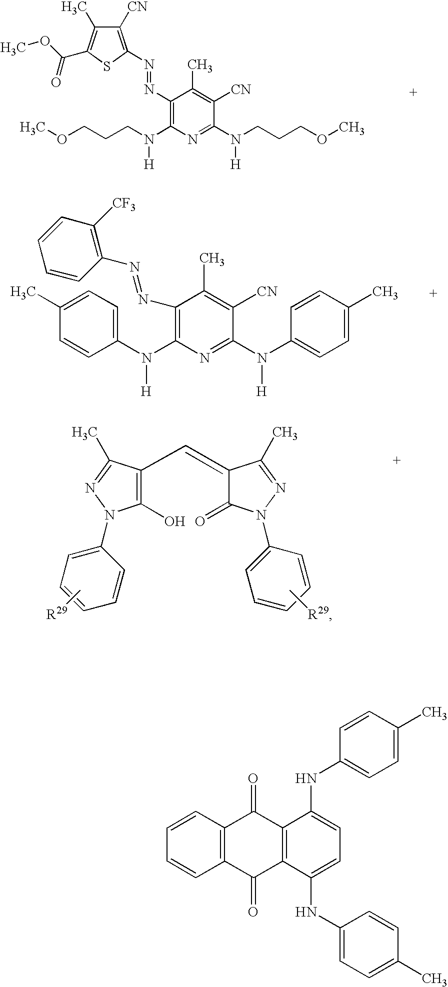

preferably in a ratio of 9.0:8.5:67.0:15.5.

In order to avoid metering problems and in order to achieve homogeneous distribution of the mixture of the invention in the plastic, many plastics processors prefer a concentrate of the additive in question, in this case, accordingly, the dye mixture in the base polymers or other carriers.

In the context of this specification such concentrates are referred to as masterbatches.

The invention accordingly also provides masterbatches containing 15%-70%, preferably 40% to 70%, by weight of the mixture of the invention and a carrier.

Preferably the amount of the carrier makes up the remainder to 95%, preferably to 98%, more particularly to 99% by weight of the masterbatch.

Suitable carriers are preferably the base polymers of the plastic itself that is to be coloured. Masterbatches of this kind are generally solid. Particularly preferred base polymers are thermoplastics, examples being vinyl polymers, polyesters, polyamides and also polyolefins, more particularly polyethylene and polypropylene, or polycarbonates.

Suitable vinyl polymers are polystyrene, styrene-acrylonitrile copolymers, styrene-butadiene copolymers, styrene-butadiene-acrylonitrile terpolymers, polymethacrylates, polyvinyl chloride, etc.

Additionally suitable are polyesters such as polyethylene terephthalates, polycarbonates and cellulose esters, for example.

Preference is given to polystyrene, styrene copolymers, polyethylene terephthalates, polycarbonates, polymethacrylates and polyamides. Particular preference is given to polystyrene, polyethylene terephthalates, polycarbonates and polymethacrylates. Especially suitable are polyethylene terephthalates.

For producing such masterbatches it is preferred to heat a base polymer or a mixture of two or more base polymers with the dye mixture of the invention, to melt the components, to carry out mixing and to convert the mixture into a pourable form, such as into granule form, for example, in the form for example of solidified droplets, beads, hollow spheres, flakes and the like.

Where the dye mixtures are to be used only after the plastics have been polymerized, they are dry-mixed or dry-ground with the plastics granules, for example, and the resulting mixture is plasticated and homogenized in screws or on mixing rolls, for example. Alternatively the mixtures can be added to the liquid and distributed homogeneously by stirring. The material pre-coloured in this way is then processed further to shaped parts in customary fashion, as for example by spinning to give bristles, filaments and so on or by extrusion or by the injection moulding process.

As carriers preference is likewise given to dispersion carriers. The use of such carriers leads preferably to liquid, pumpable masterbatches.

The invention further provides for the use of the mixture of the invention for mass-colouring plastics.

By mass colouring here is meant more particularly those processes in which the dye mixture is incorporated into the melted mass of plastic, with the aid of an extruder, for example, or in which the dye is actually added to the starting components for producing the plastic, e.g. to monomers prior to the polymerization.

Particularly preferred plastics are thermoplastics, for example vinyl polymers, polyesters, polyamides and polyolefins, more particularly polyethylene and polypropylene, or polycarbonates.

Suitable vinyl polymers are polystyrene, styrene-acrylonitrile copolymers, styrene-butadiene copolymers, styrene-butadiene-acrylonitrile terpolymers, polymethacrylates, polyvinyl chloride, etc.

Additionally suitable are polyesters such as polyethylene terephthalates, polycarbonates and cellulose esters, for example.

Preference is given to polystyrene, styrene copolymers, polyethylene terephthalates, polycarbonates, polymethacrylates and polyamides. Particular preference is given to polystyrene, polyethylene terephthalates, polycarbonates and polymethacrylates.

The high molecular mass compounds mentioned may be present individually or in mixtures, as plastic masses or melts. Particular preference is given to polyethylene terephthalate (PET).

The mixtures of the invention are employed, for example, in finely divided form, with the use of dispersants as well being possible but not mandatory. Alternatively they can also be employed, for example, in masterbatches, in granulated form, for example, in the form for example of solidified droplets, beads, hollow spheres, flakes and the like.

Since the dye mixtures are stable to polymerization catalysts, more particularly peroxides, it is also possible to add them to the monomeric starting materials for the plastics and then to carry out polymerization in the presence of polymerization catalysts. For this purpose the dye mixtures are preferably dissolved in or intimately mixed with the monomeric components.

The dye mixtures of the invention are used preferably in amounts of 0.0001% to 1%, more particularly 0.01% to 0.5%, by weight, based on the plastic or on the synthetic fibres.

Where masterbatches are used the amount is such as to result preferably in the same dye mixture fractions, based on the plastic.

By adding pigments which are insoluble in the polymers, such as titanium dioxide, for example, it is possible to obtain corresponding useful opaque colorations.

Titanium dioxide can be used in an amount of 0.01% to 10%, preferably 0.1% to 5%, by weight, based on the polymer amount.

This gives transparent or opaque, bluish red colorations having good heat stability and also good light, weather, sublimation and migration fastness. When yellow and green, and/or blue, dyes are used as well, brown colorations with the same fastness properties are obtained.

The invention therefore also provides a method of mass-colouring plastics, characterized in that the mixture according to Claim 1, preferably in the form of its masterbatch, is incorporated into the melted mass of plastic or is actually added to the starting components for the production of the plastic, prior to the polymerization.

The invention further provides plastics comprising the colour mixture of the invention. Such plastics are, more particularly, hollow articles such as bottles, more particularly beverage bottles.

This gives transparent or opaque red or brown colorations with good heat resistance and also good light, weather, sublimation and migration fastnesses.

More particularly the sublimation fastnesses and migration fastnesses of the dyes of the formula (I) according to the invention are significantly improved as compared with, for example, the dye of the formula (CI) with R²⁷═R²⁸═H (=C.I. Solvent Red 60).

The red and brown mixtures of the invention likewise exhibit good fastness properties, more particularly good sublimation and migration fastnesses. These mixtures are also notable for high solubility in the plastics, more particularly in masterbatches. As compared with brown mixtures based on C.I. Solvent Red 135, the solubility in masterbatches is significantly increased, which is unexpected owing to the relatively high molar mass of the bridged dyes of the formula (I) according to the invention.

The invention further provides compounds of the formula I

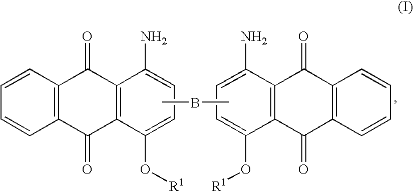

in which
—B— is —O—B¹—O— or —CH₂—B²—CH₂—,
R¹ is hydrogen, optionally substituted C₁-C₈-alkyl or optionally substituted C₃-C₈-cycloalkyl,
B¹ is a bridge of the formula

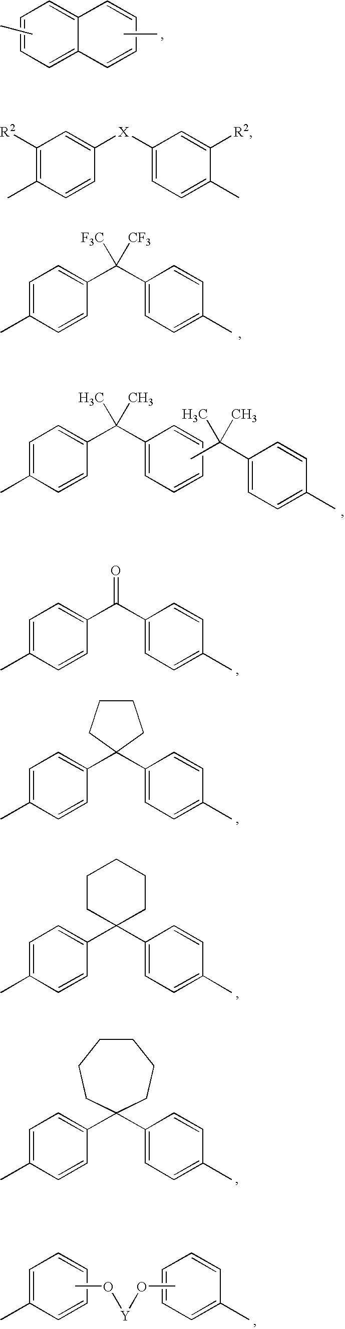

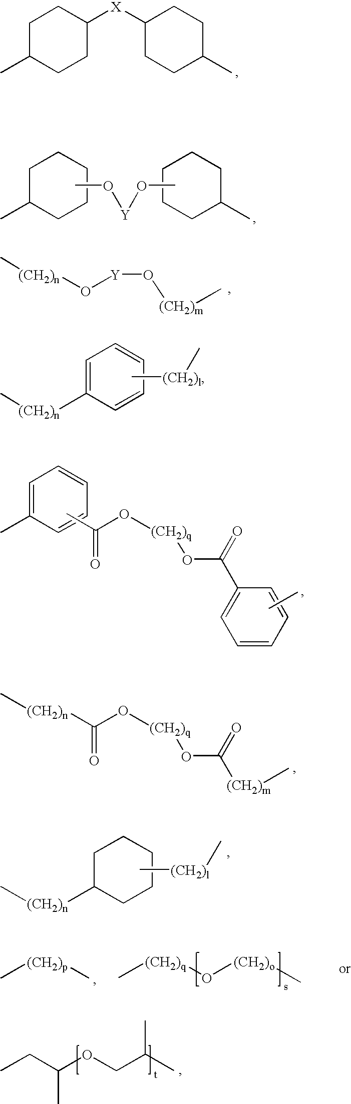

B² is a bridge of the formula

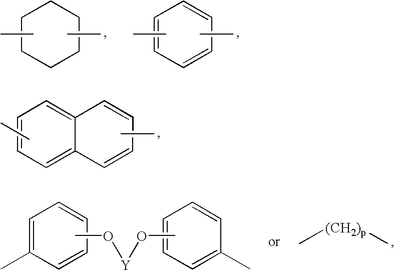

R² is methyl, ethyl, methoxy or fluorine,
X is a bridge of the formula

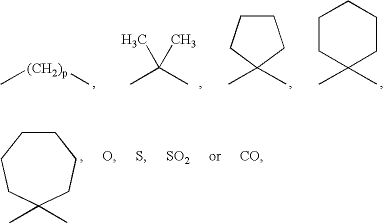

Y is a bridge of the formula

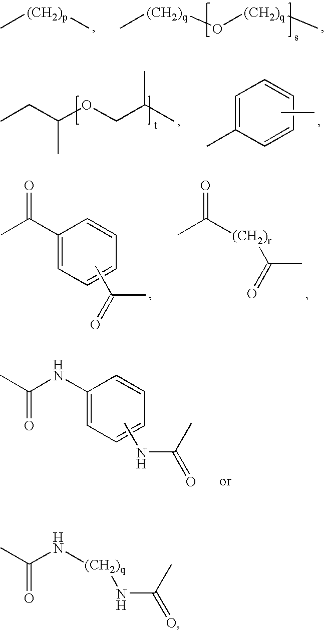

l is an integer from 0 to 8,
m, n, p and r independently of one another are an integer from 1 to 8,
q is an integer from 2 to 8 and
s and t independently of one another are an integer from 1 to 4.

The compound of the formula I preferably corresponds to the formulae (Ia) to (Ic)

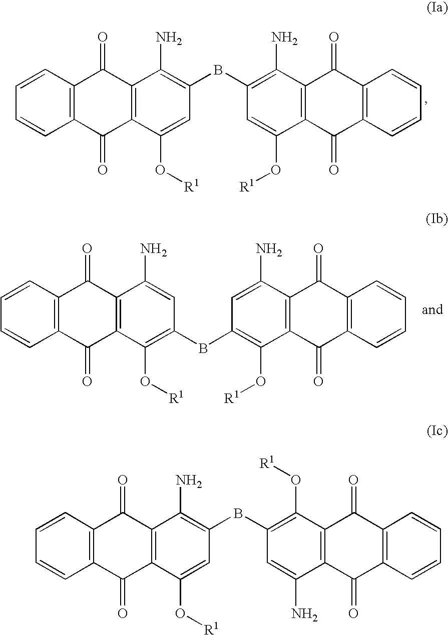

more particularly of the formula Ia.

In the case of the naphthalene structure the undefined bonds attach preferably in positions 1,5-, 1,8-, 2,6- or 2,7-. In the case of the benzene and cyclohexane structures the undefined bonds attach in positions 1,2-, 1,3- or 1,4-.

By C₁-C₈-alkyl here and below is meant linear and/or branched C₁-C₈-alkyl, e.g. methyl, ethyl, n-propyl, 2-propyl, n-butyl, 2-butyl, isobutyl, tert-butyl, pentyl, hexyl, heptyl, octyl or 2-ethylhexyl.

By substituted C₁-C₈-alkyl is meant linear and/or branched C₁-C₈-alkyl, as described above, which is substituted by at least one of the radicals fluorine, cyano, hydroxyl, C₁-C₄-alkoxy, acetoxy, propoxy, butoxy, C₁-C₄-alkoxycarbonyl, cyclopentyl, cyclohexyl, phenyl, naphthyl e.g. trifluoromethyl, perfluorobutyl, cyanomethyl, 2-cyanoethyl, 2-methoxyethyl, 2-ethoxyethyl, 2-acetoxyethyl, 2-methoxycarbonylethyl, cyclohexylmethyl, benzyl, phenethyl or phenylpropyl.

Here and below, for example, butyl is always n-butyl, 2-butyl, isobutyl or tert-butyl, unless expressly stated otherwise.

By C₃-C₈-cycloalkyl is meant, here and below, cyclopropyl, cyclobutyl, cyclopentyl, cyclohexyl, cycloheptyl or cyclooctyl. By substituted C₃-C₈-cycloalkyl is meant, for example, methylcyclohexyl.

Preferably R¹ is hydrogen. Likewise preferably R¹ is methyl.

Preferred bridges B¹ are:

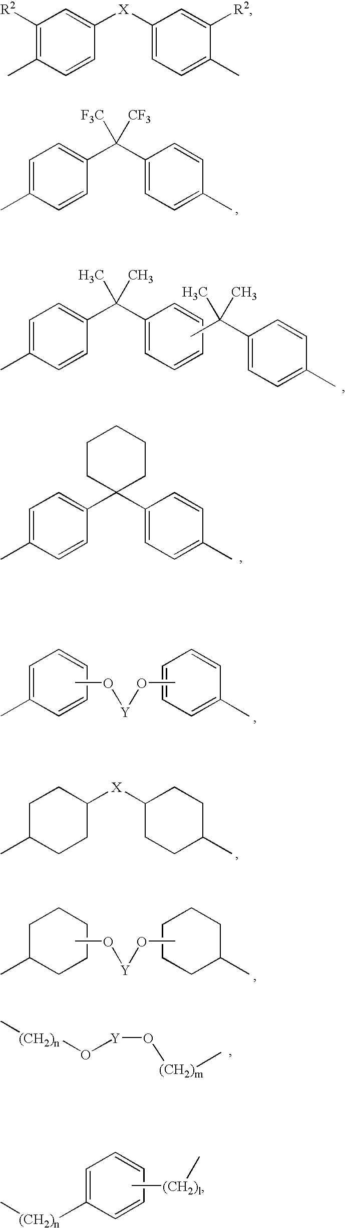

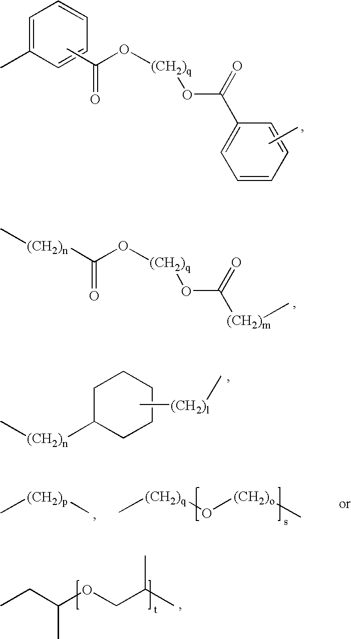

in which
R² is methyl,
X is a bridge of the formulae

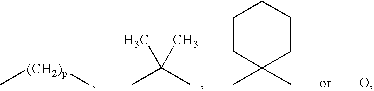

Y is a bridge of the formula

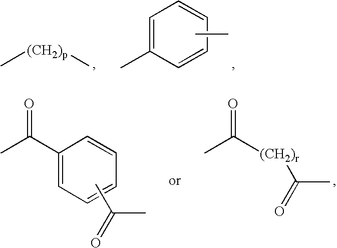

l is an integer from 0 to 4,
m, n and p independently of one another are an integer from 1 to 4,
q and r independently of one another are an integer from 2 to 6,
s and t independently of one another are an integer from 1 to 2 and
the undefined ring positions are 1,2-, 1,3- or 1,4-.

Especially preferred bridges B¹ are:

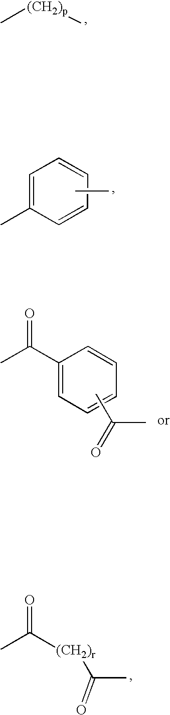

in which
Y is a bridge of the formula

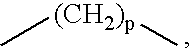

l is an integer from 1 to 2,
m, n and p independently of one another are an integer from 1 to 2,
q is an integer from 2 to 4,
s and t independently of one another are 1 and
the undefined ring positions are 1,4-.

Preferred bridges B² are:

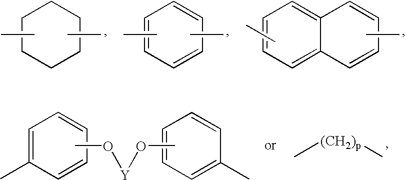

in which
Y is a bridge of the formulae

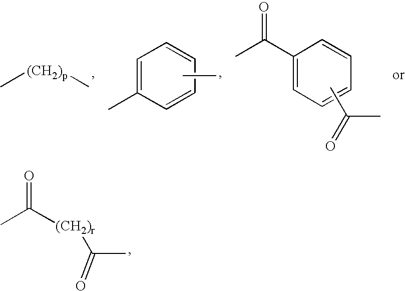

p is an integer from 1 to 6 and
r is an integer from 2 to 6.

Particularly preferred bridges B² are:

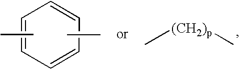

in which
p is an integer from 1 to 4.

The invention additionally provides a process for preparing the inventive dyes of the formula (I)

in which
B is a bridge —O—B¹—O—,
   characterized in that an anthraquinone compound of the formula (II)

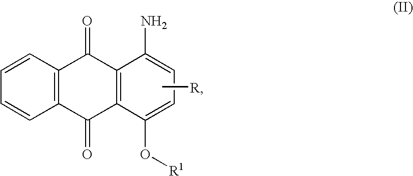

in which
R¹ is hydrogen, optionally substituted C₁-C₈-alkyl or optionally substituted C₃-C₈-cycloalkyl and
R is chlorine, bromine or phenoxy,
   is reacted with a bifunctional alcohol of the formula (III)

HO—B¹—OH (III),

in which
B¹ is a bridge of the formula

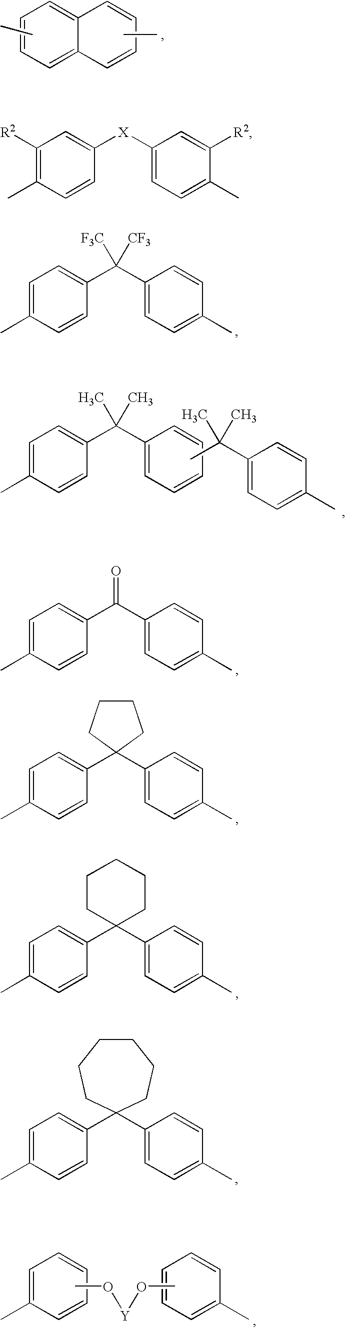

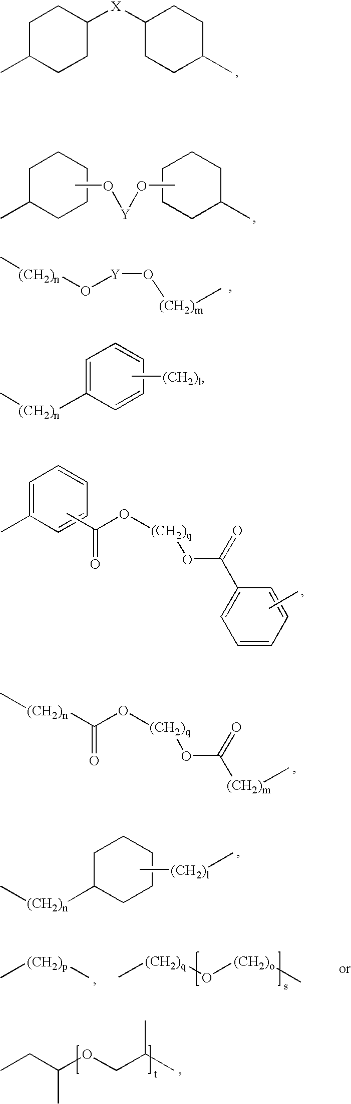

R² is methyl, ethyl, methoxy or fluorine,
X is a bridge of the formula

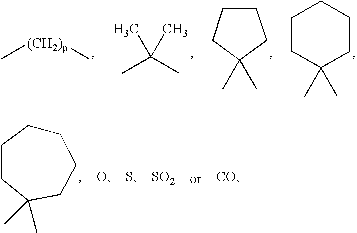

Y is a bridge of the formula

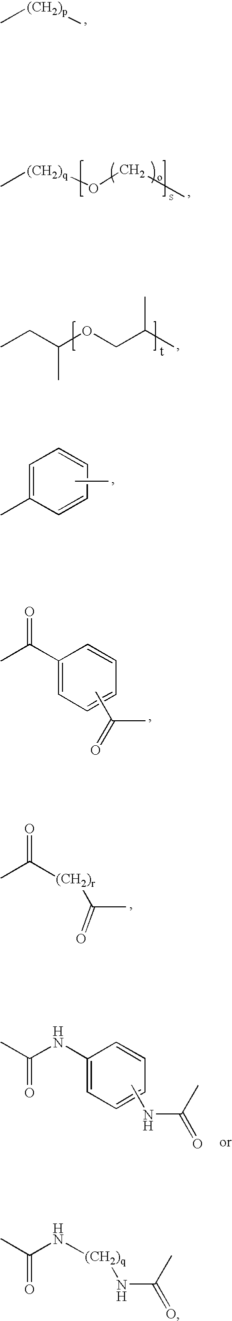

l is an integer from 0 to 8,
m, n, p and r independently of one another are an integer from 1 to 8,

q is an integer from 2 to 8 and
s and t independently of one another are an integer from 1 to 4.

The invention further provides a process for preparing the inventive dyes of the formula (I)

in which
B is a bridge —O—B¹—O— and
B¹ is a bridge of the formula

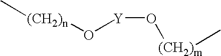

in which
Y is a bifunctional group of the formula

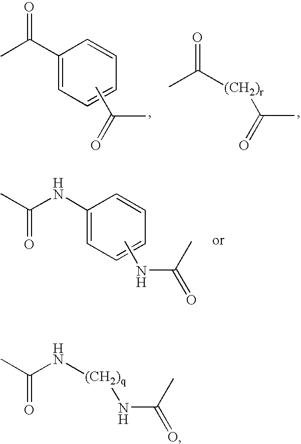

m, n and r independently of one another are an integer from 1 to 8 and n and m are alike and
q is an integer from 2 to 8,
   characterized in that an anthraquinone of the formula (IV)

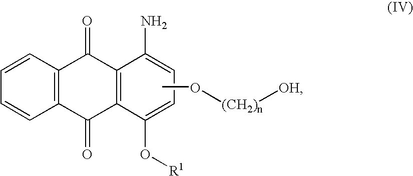

in which
R¹ is hydrogen, optionally substituted C₁-C₈-alkyl or optionally substituted C₃-C₈-cycloalkyl and
n is an integer from 1 to 8,
   is reacted with a bifunctional acid chloride or ester of the formulae (V)

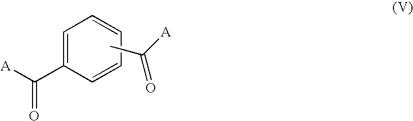

### or (VI)

A-CO—(CH₂)ᵣ—CO-A (VI)

or with a bifunctional isocyanate of the formula (VII)

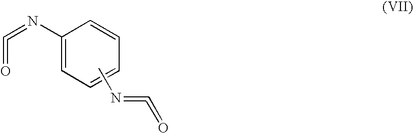

### or (VIII)

O═C═N—(CH₂)_{q}—N═C═O (VIII)

in which
A is Cl or methoxy,
r is an integer from 1 to 8 and
q is an integer from 2 to 8.

The invention further provides a process for preparing the inventive dyes of the formula (I)

in which
B is a bridge —O—B¹—O— and
B¹ is a bridge of the formula

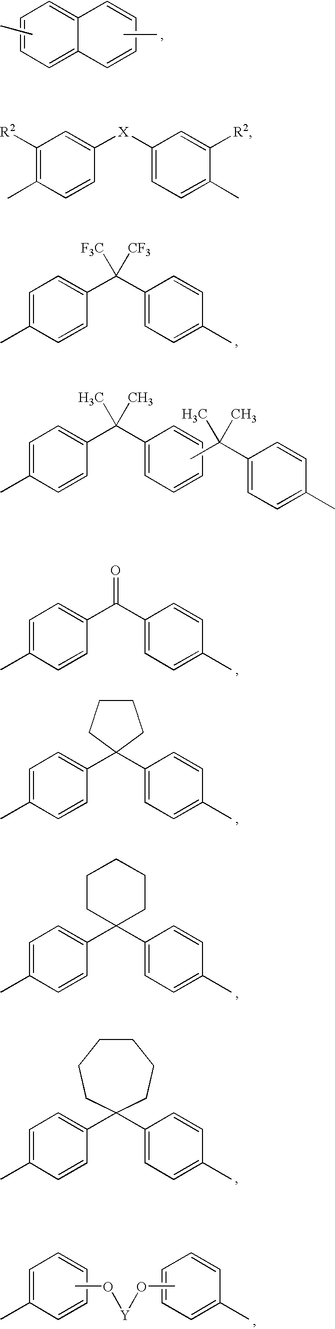

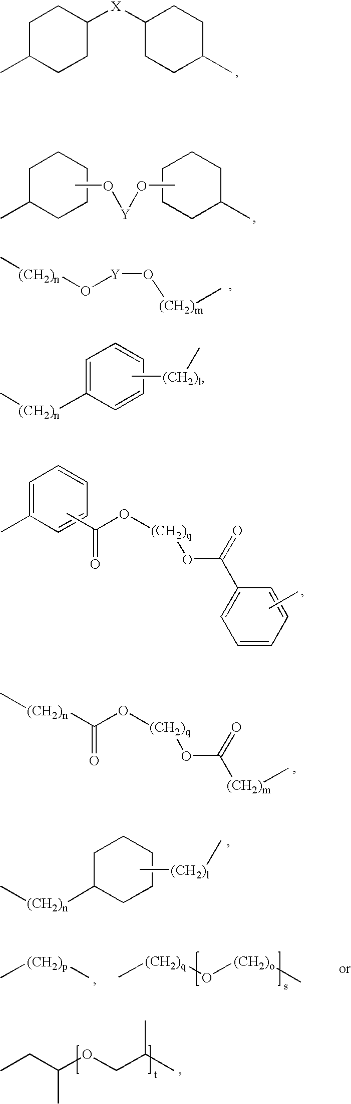

R² is methyl, ethyl, methoxy or fluorine,
X is a bridge of the formula

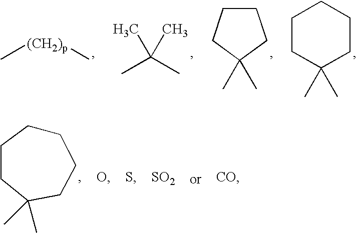

Y is a bridge of the formula

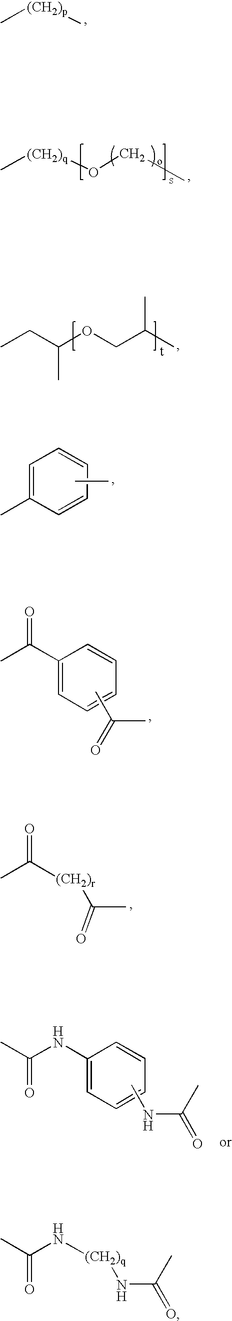

l is an integer from 0 to 8,
m, n, p and r independently of one another are an integer from 1 to 8,
q is an integer from 2 to 8 and
s and t independently of one another are an integer from 1 to 4,
   characterized in that an anthraquinone compound of the formula (II)

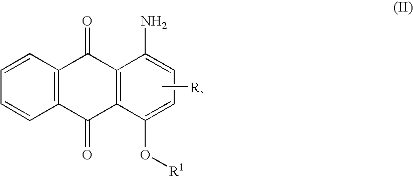

in which
R¹ is hydrogen, optionally substituted C₁-C₈-alkyl or optionally substituted C₃-C₈-cycloalkyl and
R is chlorine, bromine or phenoxy,
   is reacted with an anthraquinone compound of the formula (IX)

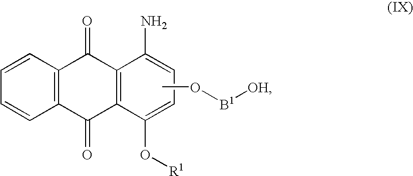

in which
R¹ is hydrogen, optionally substituted C₁-C₈-alkyl or optionally substituted C₃-C₈-cycloalkyl.

In the case of the formulae (II), (IV) and (IX), in analogy to formula (I), it is the respective isomers that are being referred to, e.g.

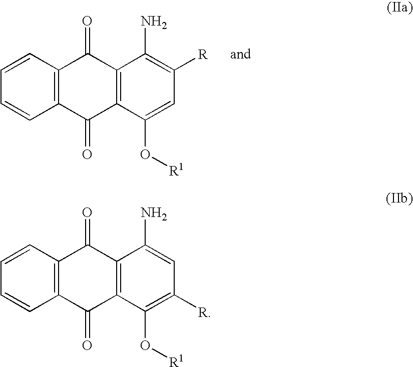

The reaction takes place, for example, in a molar ratio (II)/(III) or (IV)/(V) or (IV)/(VI) or (IV)/(VII) or (IV)/(VIII) of 2/1 or in a molar ratio (II)/(IX) of 1/1. It may also be advantageous, however, to use one of the two components in an excess relative to this ratio.

The reaction takes place advantageously in a solvent. Examples of such are dipolar aprotic solvents such as N-methyl- or -ethylpyrrolidone, dimethylformamide and aromatic solvents such as toluene, xylene, chlorobenzene, 1,2-dichlorobenzene, methyl benzoate and also heterocycles such as pyridine and quinoline.

The reaction takes place advantageously in the presence of a base. Examples of suitable bases are hydroxides, carbonates, and amines, examples being alkali metal hydroxides such as sodium or potassium hydroxide, alkali metal carbonates such as sodium or potassium carbonate, and amines such as pyridine, quinoline and triethylamine.

Phase transfer catalysts such as quaternary ammonium salts or crown ethers may likewise be added. Examples are tetrabutylammonium bromide, trimethylbenzylammonium hydroxide, tricaprylmethylammonium chloride and 18-crown-6.

The reaction takes place for example at temperatures in the range 80 to 220° C., preferably in the range 80 to 180° C., more preferably in the range 100 to 160° C.

Particular preference in the case of the reaction of the compounds of the formula (II) with those of the formula (III) is anhydrous sodium or potassium carbonate as base and N-methyl- or -ethylpyrrolidone as solvent or anhydrous sodium or potassium carbonate as base, 1,2-dichlorobenzene as solvent and 18-crown-6 or tetrabutylammonium bromide or tricaprylmethylammonium chloride as phase transfer catalyst.

When reaction is at an end the reaction products, for example, can be precipitated by adding alcohols such as methanol or ethanol, or water, or mixtures thereof, and can be isolated by filtration and, where appropriate, can be purified further.

The invention further provides a process for preparing the inventive dyes of the formula (I)

in which
B is a bridge —CH₂—B²—CH₂—,
   characterized in that a dihydroanthraquinone compound which in one tautomeric form corresponds to the formula (X)

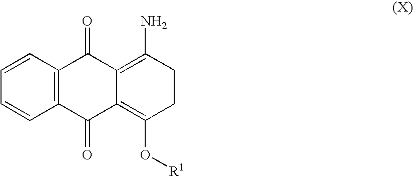

in which
R¹ is hydrogen, optionally substituted C₁-C₈-alkyl or optionally substituted C₃-C₈-cycloalkyl,
   is reacted with a bifunctional aldehyde of the formula (XI)

OCH—B²—CHO (XI),

in which
B² is a bridge of the formula

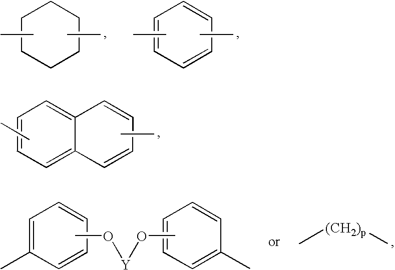

Y is a bridge of the formula

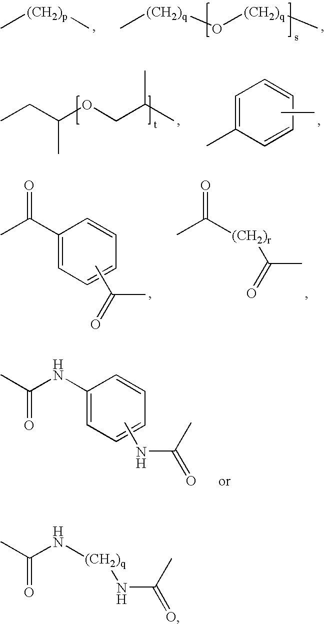

p and r independently of one another are an integer from 1 to 8,
q is an integer from 2 to 8 and
s and t independently of one another are an integer from 1 to 4.

The reaction takes place, for example, in a molar ratio (X)/(XI) of 2/1. It may also be advantageous, however, to use one of the two components in an excess relative to this ratio.

The reaction takes place advantageously in a solvent. Examples of such are C₁-C₁₀ alcohols such as methanol, ethanol, propanol, 2-propanol, butanol, pentanol, hexanol, benzyl alcohol, dipolar aprotic solvents such as N-methyl- or -ethylpyrrolidone, dimethylformamide and aromatic solvents such as toluene, xylene, chlorobenzene, 1,2-dichlorobenzene, methyl benzoate and also heterocycles such as pyridine and quinoline.

The reaction takes place advantageously in the presence of acids and/or bases. Examples of suitable acids are hydrochloric acid, benzenesulphonic acid, toluenesulphonic acid, acetic acid, methanesulphonic acid. Examples of suitable bases are amines such as piperidine, morpholine, piperazine, pyridine, quinoline and triethylamine. Likewise advantageous is the combination of acids and bases, for example acetic acid and piperidine.

The reaction takes place for example at temperatures in the range 40 to 180° C., preferably in the range 70 to 120° C.

When reaction is at an end it is possible, for example, for the reaction products to be precipitated, where appropriate, by addition of alcohols such as methanol or ethanol or of water, or mixtures thereof, and to be isolated by filtration and, where appropriate, to be purified further.

The invention further provides masterbatches containing 15% to 70%, preferably 40% to 70%, by weight of the inventive dye of the formula I and a carrier.

The invention further provides for the use of the compounds of the formula I according to the invention for the mass colouring of plastics.

The invention further provides for the use of the compound of the formula I of the invention or of the mixtures of the invention for colouring synthetic fibres, preferably in dispersed form.

### EXAMPLES

### Example 1

2.70 g of cyclohexylidenebisphenol were stirred under a nitrogen atmosphere in 20 ml of N-methylpyrrolidone with 4 ml of 50 percent strength potassium hydroxide solution at 100° C. for 3 h. Subsequently 6.40 g of 1-amino-2-bromo-4-hydroxyanthraquinone were introduced. The mixture was stirred at 120° C. for 2 h, cooled to 80° C., diluted with 15 ml of methanol and, after cooling to room temperature, subjected to suction filtration. The solid product was washed with 10 ml of methanol and 100 ml of hot water and dried under reduced pressure at 80° C. This gave 3.90 g (52% of theory) of a red powder of the formula

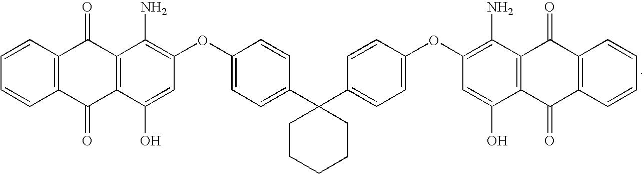

UV/VIS (NMP): λₘₐₓ=523, 561 nm, ε=24880 l mol⁻¹ cm⁻¹ (at 523 nm).

### Example 2

In the same way as in Example 1, but using 3.5 g of 4,4-(1,3-phenylenediisopropylidene)-bisphenol, 4.80 g (57.6% of theory) were obtained of a red powder of the formula

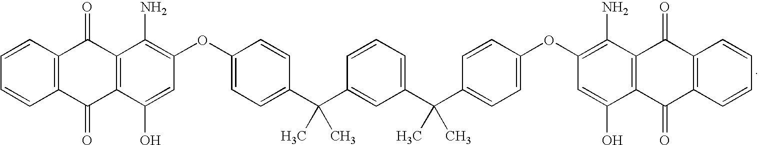

UV/VIS (NMP): λₘₐₓ=523, 560 nm. ε=20850 l mol⁻¹ cm⁻¹ (at 523 nm).

### Example 3

2.30 g of 2,2-bis(4-hydroxy-3-methylphenyl)propane were stirred under a nitrogen atmosphere in 15 ml of N-methylpyrrolidone with 4 ml of 50 percent strength potassium hydroxide solution at 100° C. for 3 h. Subsequently 5.71 g of 1-amino-2-bromo-4-hydroxyanthraquinone were introduced. The mixture was stirred at 120° C. for 7 h, cooled to 80° C., diluted with 15 ml of methanol and, after cooling to room temperature, subjected to suction filtration. The solid product was washed with 10 ml of methanol and 100 ml of hot water and dried under reduced pressure at 80° C. This gave 1.07 g (16% of theory) of a red powder of the formula

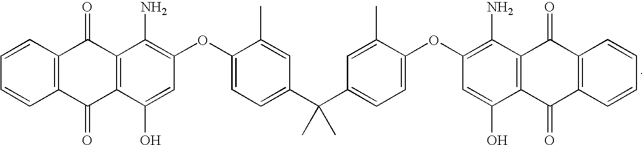

UV/VIS (NMP): λₘₐₓ=522, 558 nm, ε=23970 l mol⁻¹ cm⁻¹ (at 522 nm).

### Example 4

Under a nitrogen atmosphere 2.50 g of 1,4-bis(2-hydroxyethoxy)benzene, 1.42 g of potassium hydroxide and 8.36 g of 1-amino-2-phenoxy-4-hydroxyanthraquinone were introduced into 20 ml of N-ethylpyrrolidone and the mixture was stirred at 130° C. for 11 h. After cooling to 80° C. it was diluted with 60 ml of methanol and after cooling to room temperature it was subjected to suction filtration. The solid product was washed with 10 ml of methanol and 100 ml of hot water and then dried under reduced pressure at 80° C. This gave 6.80 g (80% of theory) of a red powder of the formula

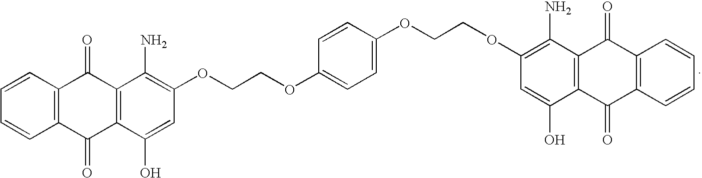

UV/VIS (NMP): λₘₐₓ=518, 555 nm, ε=24420 l mol⁻¹ cm⁻¹ (at 518 nm).

### Example 5

Under a nitrogen atmosphere 2.50 g of 2,7-dihydroxynaphthalene, 3.31 g of anhydrous sodium carbonate, 0.83 g of 18-crown-6 and 9.93 g of 1-amino-2-bromo-4-hydroxyanthraquinone were introduced into 25 ml of 1,2-dichlorobenzene and the mixture was stirred at 165° C. for 4 h. After cooling to 80° C. it was diluted with 60 ml of methanol, stirred at 60° C. for 1 h and after cooling to room temperature it was subjected to suction filtration. The solid product was washed with 10 ml of methanol and 100 ml of hot water and then dried under reduced pressure at 80° C. This gave 7.51 g (76% of theory) of a red powder of the formula

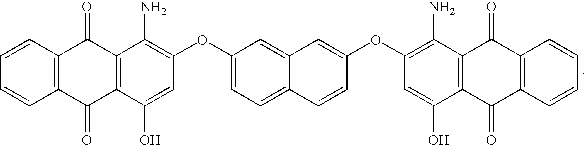

UV/VIS (NMP): λₘₐₓ=525, 560 nm, ε=26710 l mol⁻¹ cm⁻¹ (at 525 nm).

### Example 6

In the same way as in Example 5, using 2.50 g of 2,6-dihydroxynaphthalene, 8.00 g (80.8% of theory) were obtained of a red powder of the formula

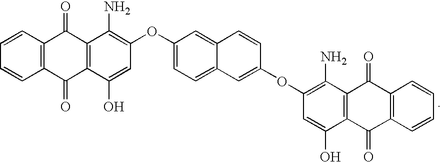

UV/VIS (IMP): λₘₐₓ=525, 561 mm ε=23870 l mol⁻¹ cm⁻¹ (at 525 nm).

### Example 7

Under a nitrogen atmosphere 2.50 g of 1,4-butanediol and 5.88 g of sodium carbonate were stirred in 35 ml of N-ethylpyrrolidone at 150° C. for 3 h. Then 18.4 g of 1-amino-2-phenoxy-4-hydroxyanthraquinone were introduced and the mixture was stirred at 160° C. for 24 h. After cooling to 80° C. it was diluted with 60 ml of methanol, stirred at 60° C. for 1 h and, after cooling to room temperature, was subjected to suction filtration. The solid product was washed with 10 ml of methanol and 100 ml of hot water and then dried under reduced pressure at 80° C. This gave 8.53 g of a red powder. This powder was purified by boiling twice in 100 ml of toluene for 2 h, isolating by suction filtration while hot, and washing with 250 ml of hot toluene and then 100 ml of hot methanol. Finally the product was washed with 100 ml of hot water and dried under reduced pressure at 80° C. This gave 5.42 g (35% of theory) of a red powder of the formula

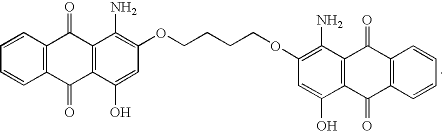

UV/VIS (NMP): λₘₐₓ=518, 556 nm, ε=14740 l mol⁻¹ cm⁻¹ (at 518 nm).

### Example 8

a) 2.24 g of potassium hydroxide were dissolved in 62 g of ethylene glycol. 9.9 g of 1-amino-2-phenoxy-4-hydroxyanthraquinone were introduced and the mixture was stirred under a nitrogen atmosphere at 120° C. for 6 h. After cooling to room temperature it was subjected to suction filtration and the solid product was washed with 20 ml of ethylene glycol and 100 ml of water and dried under reduced pressure at 80° C. This gave 7.7 g (86% of theory) of a red powder of the formula

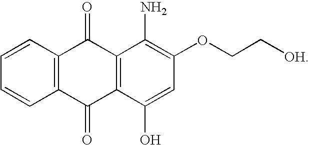

b) 2.0 g of the product from a) were introduced under a nitrogen atmosphere in 25 ml of pyridine, 0.68 g of terephthaloyl dichloride was added and the mixture was stirred at reflux for 7 h. After cooling to room temperature it was subjected to suction filtration and the solid product was washed with 10 ml of methanol and 100 ml of hot water and dried under reduced pressure at 80° C. This gave 1.85 g (76% of theory) of a red powder of the formula

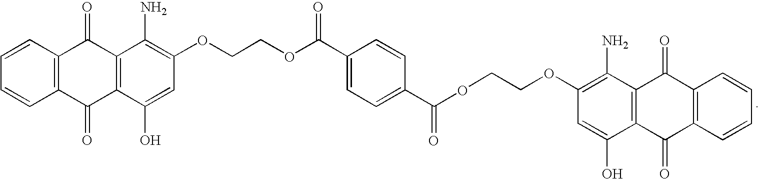

UV/VIS (NMP): λₘₐₓ=518, 555 nm.

### Example 9

2.53 g of 1,4-cyclohexanedimethanol and 3.71 g of anhydrous sodium carbonate were stirred in 30 ml of N-ethylpyrrolidone under a nitrogen atmosphere at 120° C. for 3 h. 11.49 g of 1-amino-2-phenoxy-4-hydroxyanthraquinone were introduced and then the mixture was stirred at 160° C. for 15 h. After cooling to 100° C. it was diluted with 60 ml of methanol and cooled to room temperature. The solid product was isolated by suction filtration, washed with 10 ml of methanol and 100 ml of hot water and dried under reduced pressure at 80° C. The crude product was boiled in 100 ml of toluene for 3 h with stirring, isolated by suction filtration while hot, washed with 10 ml of hot toluene and 30 ml of methanol and dried under reduced pressure at 80° C. This gave 2.71 g (25% of theory) of a red powder of the formula

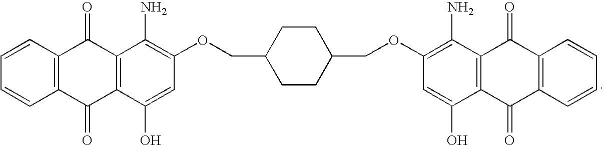

UV/VIS (NMP): λₘₐₓ=519, 555 nm, ε=21960 l mol⁻¹ cm⁻¹ (at 519 nm).

### Example 10

In the same way as in Example 1, using 2.3 g of 2,2-bis(4-hydroxyphenyl)propane and 6.69 g of 1-amino-2-bromo-4-methoxyanthraquinone, 4.73 g (64.3% of theory) were obtained of the red dye of the formula

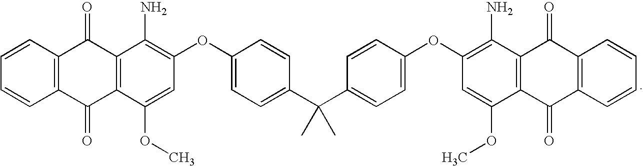

UV/VIS (NMP): λₘₐₓ=521, 552 nm, ε=13450 l mol⁻¹ cm⁻¹ (at 521 nm).

### Example 11

In the same way as in Example 1, using 2,2-bis(4-hydroxyphenyl)propane and 1-amino-3-bromo-4-hydroxyanthraquinone, the dye of the formula

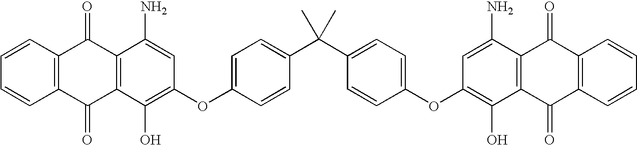

was obtained.

### Example 12

6.70 g of terephthalaldehyde, 24.1 g of 1-amino-4-hydroxydihydroanthraquinone, 2.55 g of piperidine and 1.80 g of glacial acetic acid were stirred under a nitrogen atmosphere in 150 ml of N-methylpyrrolidone at 150° C. for 8 h. After cooling to room temperature, the violet suspension was subjected to suction filtration and the solid product was washed with 40 ml of N-methylpyrrolidone, 100 ml of methanol and 300 ml of hot water. Drying at 80° C. under reduced pressure gave 15.8 g (54% of theory) of a violet powder of the formula

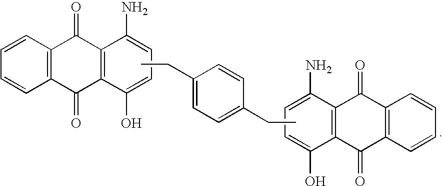

UV/VIS (NMP): λₘₐₓ=538, 574 nm.

### COLOURING EXAMPLES

a) 100 g of polystyrene granules and 0.02 g of the dye from Example 4 are mixed intensely in a drum mixer for 15 min. The dry-stained granules are processed on a screw injection moulding machine at 240° C. This gives transparent bluish red sheets of very good light fastness and migration fastness.

Instead of polystyrene polymer it is also possible to use copolymers with butadiene and acrylonitrile.

If an additional 0.5 g of titanium dioxide is added, strongly opaque colorations are obtained.

b) 0.025 g of the dye from Example 4 are mixed with 100 g of a transparent polyethylene terephthalate grade and the mixture is homogenized in a twin-screw extruder at 280° C. This gives a transparent bluish red coloration with good light fastness and migration fastness. After subsequent granulation the coloured plastic can be processed in accordance with the typical methods of thermoplastic shaping.

Operating with addition of 1% of titanium dioxide produces an opaque coloration.

c) 100 g of a commercially customary polycarbonate are dry-mixed in granule form with 0.03 g of the dye from Example 4. The granules dusted in this way are homogenized in a twin-screw extruder at 290° C. This gives a transparent bluish red coloration with good light fastness and migration fastness. The coloured polycarbonate is extruded as a strand from the extruder and processed to granules. The granules can be processed by the typical methods of processing thermoplastic compounds.

Operating with addition of 1% of titanium dioxide produces an opaque coloration.

d) A similar procedure is operated with a styrene-acrylonitrile copolymer, but with homogenization at 190° C.

e) 0.05 g of tert-dodecyl mercaptan and 0.05 g of the dye from Example 4 are dissolved in 98.9 g of styrene. This solution is dispersed in a solution of 200 g of demineralized water, 0.3 g of partially hydrolysed polyvinyl acetate (e.g. Mowiol® 50/88) and 0.05 g of dodecylbenzenesulphonate. Following addition of 0.1 g of dibenzoyl peroxide in solution in 1 g of styrene, the dispersion is heated to 80° C. with vigorous stirring and the polymerization is initiated. Employing the following polymerization conditions—4 h at 80° C., 2 h at 90° C., 3 h at 110° C., 2 h at 13° C.—produces the polymer in a yield of 98% of theory. The polymer is obtained in the form of beads which, depending on stirring conditions, have a diameter of 0.1 to 1.5 mm (D₅₀). The polymer is separated from the liquid phase by filtration and is dried at 110° C. to a residual moisture content of 0.5%. After melting (hot roll), 0.5% of zinc stearate and 0.2% of Ionol are mixed in and the polymer is granulated.

The bluish red coloured polymer can be processed by the typical methods of thermoplastic shaping, such as by injection moulding, for example, to give bluish red transparent mouldings.

In a similar way the dyes of Examples 1-3 and 5-11 and also the dyes and mixtures in the table below can be employed. The results are red or brown colorations having good fastness properties.

### MIXING EXAMPLES

The dyes used were as follows:

(1)=formula (Ia) with R¹=H and —B—=

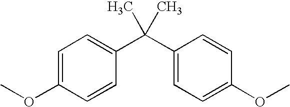

(2)=formula (Ia) with R¹=H and —B—=

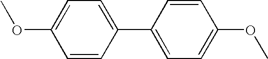

(3)=Example 4=formula (Ia) with R¹=H and —B—=

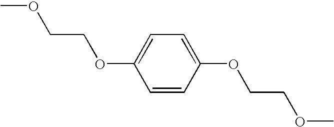

(4)=formula (L) with R¹⁰=COOCH₃ and R¹¹=H
(5)=formula (C) with R²⁰=COOCH₃, R²¹=R²³=CH₃, R²²=R²⁴=CN, R²⁵=—CH₂CH₂CH₂OCH₃, R²⁶=H.
(6)=formula (LIII)

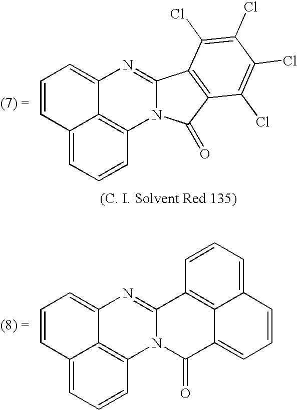

(9)=formula (CI) with R²⁷=R²⁸=H
(10)=formula (CCI)
(11)=formula (CCII) with R²⁹=H
(12)=formula (CCIII) with R³⁰=CH₃, R³¹=CN, R³²=ethyl, R³³=p-positioned cyclohexyl
(13)=formula (CCLI) with R³⁴=R³⁵=H, R³⁶=R³⁷=p-positioned CH₃

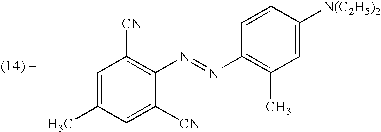

(15)=Example 1=formula (Ia) with R¹=H and —B—=

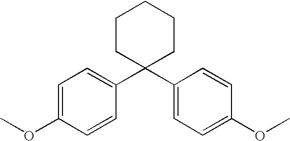

### DYE EXAMPLES OF THE MIXTURES

| | CIE colour coordinates, | | | | |
|---|---|---|---|---|---|
| | transmission measurement | | | | |
| PET transparent 2 mm | Illum. | Angle | L* | a* | b* |
| (7) (comparative) | D65 | 10° | 54.1 | 68.9 | 48.9 |
| (8) (comparative) | D65 | 10° | 52.8 | 66.5 | 70.5 |
| (14) (comparative) | D65 | 10° | 37.2 | 70.9 | 56.8 |
| (5) (comparative) | D65 | 10° | 38.4 | 74.4 | 29.6 |
| Amber 1 a (comparative) | D65 | 10° | 61.1 | 14.9 | 59.3 |
| Amber 1 b | D65 | 10° | 63.0 | 13.6 | 59.0 |
| Amber 1 c | D65 | 10° | 62.3 | 13.9 | 58.6 |
| Amber 1 d | D65 | 10° | 61.9 | 14.3 | 59.3 |
| Amber 2 a (comparative) | D65 | 10° | 59.7 | 10.8 | 46.0 |
| Amber 2 b | D65 | 10° | 60.9 | 10.1 | 45.3 |
| Amber 2 c | D65 | 10° | 61.6 | 10.7 | 45.4 |
| Amber 2 d | D65 | 10° | 58.7 | 11.7 | 46.0 |
| Amber 3 a (comparative) | D65 | 10° | 77.3 | 3.2 | 43.6 |
| Amber 3 b | D65 | 10° | 77.0 | 2.8 | 45.5 |
| Amber 3 c (comparative) | D65 | 10° | 62.8 | 13.7 | 58.5 |
| Amber 3 d | D65 | 10° | 62.4 | 14.0 | 59.4 |
| Amber 3 e (comparative) | D65 | 10° | 35.5 | 28.3 | 55.4 |
| Amber 3 f | D65 | 10° | 35.7 | 29.4 | 55.1 |
| Amber 4 a (comparative) | D65 | 10° | 74.6 | 3.7 | 33.6 |
| Amber 4 b | D65 | 10° | 74.9 | 3.5 | 33.6 |
| Amber 4 c (comparative) | D65 | 10° | 60.8 | 10.1 | 45.3 |
| Amber 4 e (comparative) | D65 | 10° | 30.0 | 21.2 | 44.7 |
| Amber 4 f | D65 | 10° | 30.6 | 22.8 | 44.7 |
| (5) (comparative) | D65 | 10° | 43.8 | 80.3 | 3.4 |
| (1) | D65 | 10° | 52.1 | 80.4 | −13.6 |
| (2) | D65 | 10° | 50.2 | 81.0 | −12.1 |
| (3) | D65 | 10° | 55.7 | 77.2 | −4.9 |
| Red 1 a | D65 | 10° | 42.5 | 73.2 | 72.5 |
| Red 1 b | D65 | 10° | 50.4 | 71.2 | 54.5 |
| Red 1 c | D65 | 10° | 62.2 | 56.8 | 32.7 |
| (7) (comparative) | D65 | 10° | 54.0 | 69.0 | 48.9 |
| Red 2 a | D65 | 10° | 48.2 | 72.1 | 51.3 |
| Red 2 b | D65 | 10° | 46.6 | 72.4 | 51.1 |
| Red 2 c | D65 | 10° | 51.9 | 69.8 | 59.9 |
| Red 2 d | D65 | 10° | 47.5 | 71.3 | 59.1 |
| Red 2 e | D65 | 10° | 46.1 | 71.7 | 57.9 |
| Red 2 f | D65 | 10° | 51.1 | 69.6 | 66.0 |
| Amber 5 a (comparative) | D65 | 10° | 61.7 | 14.5 | 69.2 |
| Amber 5 b | D65 | 10° | 61.0 | 13.1 | 50.9 |
| Amber 5 c | D65 | 10° | 60.8 | 13.2 | 48.8 |
| Amber 5 d | D65 | 10° | 62.6 | 13.3 | 55.2 |
| Amber 5 e | D65 | 10° | 57.0 | 24.9 | 70.3 |
| Amber 5 f | D65 | 10° | 56.8 | 24.2 | 66.7 |
| Amber 5 g | D65 | 10° | 59.0 | 23.3 | 73.1 |

| | Dye/100 g PET [g] | | | | |
|---|---|---|---|---|---|
| | (7) | (8) | (14) | | |
| PET transparent | compar- | compar- | compar- | | |
| 2 mm | ative | ative | ative | (5) | Total dye |
| (7) (comparative) | 0.05000 | | | | 0.050000 |
| (8) (comparative) | | 0.05000 | | | 0.050000 |
| (14) (comparative) | | | 0.05000 | | 0.050000 |
| (5) (comparative) | | | | 0.05000 | 0.050000 |

| | Dye/100 g PET [g] | | | | | | |
|---|---|---|---|---|---|---|---|
| PET | | (5) + (4) | | | | | |
| transparent | (7) | 50.8/ | | | | | |
| 2 mm | comparative | 49.2 | (13) | (10) | (11) | (12) | Total dye |
| Amber 1 a | 0.01210 | | 0.00315 | 0.00475 | | | 0.020000 |
| (comparative) | | | | | | | |
| Amber 1 b | | 0.00370 | 0.00314 | 0.00492 | | | 0.011760 |
| Amber 1 c | | 0.00350 | 0.00310 | | 0.01320 | | 0.019800 |
| Amber 1 d | | 0.00370 | 0.00310 | | | 0.00470 | 0.011500 |
| | | | | | | | 0.000000 |
| Amber 2 a | 0.01230 | | 0.00460 | 0.00310 | | | 0.020000 |
| (comparative) | | | | | | | |
| Amber 2 b | | 0.00370 | 0.00400 | 0.00290 | | | 0.010601 |
| Amber 2 c | | 0.00340 | 0.00360 | | 0.00830 | | 0.015300 |
| Amber 2 d | | 0.00370 | 0.00410 | | | 0.00300 | 0.010800 |
| Amber 3 a | 0.00605 | | 0.00157 | 0.00238 | | | 0.010000 |
| (comparative) | | | | | | | |
| Amber 3 b | | 0.00019 | 0.00016 | 0.00025 | | | 0.000588 |
| Amber 3 c | 0.01210 | | 0.00315 | 0.00475 | | | 0.020000 |
| (comparative) | | | | | | | |
| Amber 3 d | | 0.00370 | 0.00314 | 0.00492 | | | 0.011760 |
| Amber 3 e | 0.03024 | | 0.00787 | 0.01189 | | | 0.050000 |
| (comparative) | | | | | | | |
| Amber 3 f | | 0.00925 | 0.00785 | 0.01230 | | | 0.029400 |
| Amber 4 a | 0.00615 | | 0.00229 | 0.00156 | | | 0.010000 |
| (comparative) | | | | | | | |
| Amber 4 b | | 0.00185 | 0.00200 | 0.00145 | | | 0.005300 |
| Amber 4 c | 0.01229 | | 0.00459 | 0.00312 | | | 0.020000 |
| (comparative) | | | | | | | |
| Amber 4 d | | 0.00370 | 0.00400 | 0.00290 | | | 0.010600 |
| Amber 4 e | 0.03070 | | 0.01150 | 0.00780 | | | 0.050000 |
| (comparative) | | | | | | | |
| Amber 4 f | | 0.00925 | 0.01000 | 0.00725 | | | 0.026500 |
| PET | Dye/100 g PET [g] | | | | | | |
| transparent | (7) | | | | | | |
| 2 mm | comparative | (5) | (1) | (2) | (3) | (4) | Total dye |
| (5) | | 0.02500 | | | | | 0.025000 |
| (comparative) | | | | | | | |
| (1) | | | 0.02500 | | | | 0.025000 |
| (2) | | | | 0.02500 | | | 0.025000 |
| (3) | | | | | 0.02500 | | 0.025000 |
| Red 1 a | | 0.02540 | | | | 0.02460 | 0.050000 |
| Red 1 b | | | 0.02700 | | | 0.00550 | 0.032500 |
| Red 1 c | | | 0.01350 | | | 0.00280 | 0.016300 |
| (7) | 0.05000 | | | | | | 0.050000 |
| (comparative) | | | | | | | |
| Red 2 a | | | 0.03000 | | | 0.01800 | 0.048000 |
| Red 2 b | | | | 0.03000 | | 0.01800 | 0.048000 |
| Red 2 c | | | | | 0.03000 | 0.01800 | 0.048000 |
| Red 2 d | | | 0.03000 | | | 0.00600 | 0.036000 |
| Red 2 e | | | | 0.03000 | | 0.00600 | 0.036000 |
| Red 2 f | | | | | 0.03000 | 0.00600 | 0.036000 |

| PET | Dye/100 g PET [g] | | | | | | | | |
|---|---|---|---|---|---|---|---|---|---|
| transparent | (7) | | | | | | | | |
| 2 mm | comparative | (1) | (2) | (3) | (13) | (4) | (6) | (10) | Total dye |
| Amber 5 a | 0.01210 | | | | 0.00315 | | | 0.00475 | 0.020000 |
| (comparative | | | | | | | | | |
| Amber 5 b | | 0.00570 | | | 0.00320 | | 0.00340 | 0.00480 | 0.017100 |
| Amber 5 c | | | 0.00530 | | 0.00320 | | 0.00310 | 0.00480 | 0.016400 |
| Amber 5 d | | | | 0.00600 | 0.00320 | | 0.00360 | 0.00480 | 0.017600 |
| Amber 5 e | | 0.00530 | | | 0.00320 | 0.00320 | | 0.00480 | 0.016500 |
| Amber 5 f | | | 0.00500 | | 0.00320 | 0.00300 | | 0.00480 | 0.016000 |
| Amber 5 g | | | | 0.00550 | 0.00320 | 0.00330 | | 0.00480 | 0.016800 |

### Solutions of the Mixtures

The maximum solubility of the dyes was determined in methyl benzoate. These saturated solutions were finally admixed with the stated amount of the orange dye (4) (=formula (L) with R¹⁰=COOCH₃ and R¹¹=H). This dye likewise went completely into solution.

### Maximum Solubility of the Dyes in Methyl Benzoate and Spectral Data

| | Solubility | Absorption | |
|---|---|---|---|
| Dye | g/100 ml | maximum | E11 |
| (7) | 0.107 | 496 nm | 0.17 |
| (5) | 0.37 | 536 nm | 3.2 |
| (15) | 0.57 | 523, 558 nm | 1.8 |
| (1) | 0.55 | 523, 558 nm | 1.75 |
| (2) | 0.05 | 524, 560 nm | 0.06 |
| (4) | 1.64 | 471 nm | 19 |

E11 = Extinction (absorbance) of 1 g of this solution in 100 ml of N-methyl-pyrrolidone as measured in a 1 cm cell

### Solutions of the Red Mixtures in Methyl Benzoate and Spectral Data

| | Amount | | Amount | Absorption | |
|---|---|---|---|---|---|
| Dye 1 | g/100 ml | Dye 2 | g/100 ml | maximum | E11 |
| (7) | 0.107 | — | — | 496 nm | 0.17 |
| (5) | 0.37 | (4) | 0.346 | 490 nm | 5 |
| (15) | 0.57 | (4) | 0.6 | 477, 557 (sh) | 7.45 |
| | | | | nm | |
| (15) | 0.57 | (4) | 0.383 | 481, 517 | 5 |
| | | | | (sh), 558 nm | |
| (15) | 0.57 | (4) | 0.15 | 489, 519, | 2.6 |
| | | | | 559 nm | |
| (1) | 0.55 | (4) | 0.583 | 475, 556 (sh) | 6.65 |
| | | | | nm | |
| (1) | 0.55 | (4) | 0.138 | 489, 519, | 2.8 |
| | | | | 558 nm | |
| (2) | 0.05 | (4) | 0.07 | 473, 557 (sh) | 0.7 |
| | | | | nm | |
| (2) | 0.05 | (4) | 0.035 | 492, 521, | 0.66 |
| | | | | 560 nm | |

E11 = Extinction (absorbance) of 1 g of this solution in 100 ml of N-methyl-pyrrolidone as measured in a 1 cm cell
(sh) = shoulder

The spectral data of the solution were investigated. It is found that, based on the maximum achievable colour strength (=absorbance) with a saturated solution of the comparative dye (7)

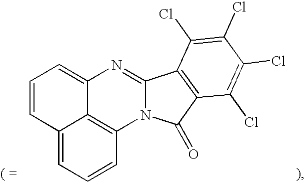

the solutions of the inventive red dyes and red mixtures are up to more than 80 times stronger in colour.

### Extrusion Trials

The extrusion trials were carried out with PET as plastic and
a) with the comparative dye (7)
b) with a 50/50 mixture of the red dye (5) and the orange dye (4).

For the trials a ZSE 18 HP extruder from Leistritz was used.

The speed was held constant at 600 rpm and the jacket temperature of the barrel at 245° C.

The PET/dye mixture was metered via a Brabender DS28 metering unit with a single screw with a speed of 25 rpm

The PET grade used was Voridian 9921W.

As is apparent from the table below, the melt pressure rises significantly from 12 to 16 bar, using dye (7), when the concentration of the dye is raised from 20% to 30%. In contrast, the melt pressure when using a dye mixture composed of equal parts of dye (5) and (4) and having a total dye content of 30%, at 4 bar, is much lower and, surprisingly, does not rise further even with an overall dye concentration of up to 45%. Accordingly a much higher loading of the batch is possible.

Pressure (bar)

| Dye | Mixture of dyes (5) and (4) | |
|---|---|---|
| content | 1:1 | Dye (7) |
| 20 | 7 | 12 |
| 30 | 4 | 16 |
| 40 | 4 | — |
| 45 | 4 | — |

1. Mixtures comprising at least one red dye of formulae (I), (C) or (CI) 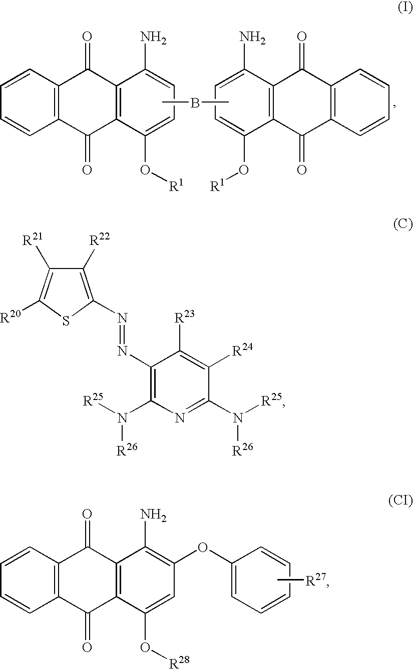
wherein
R¹ is hydrogen, unsubstituted or substituted C₁-C₈-alkyl or unsubstituted or substituted C₃-C₈-cycloalkyl,
B is a bridge of the formulae —O—B¹—O— or —CH₂—B²—CH₂—,
B¹ is a bridge of the formula 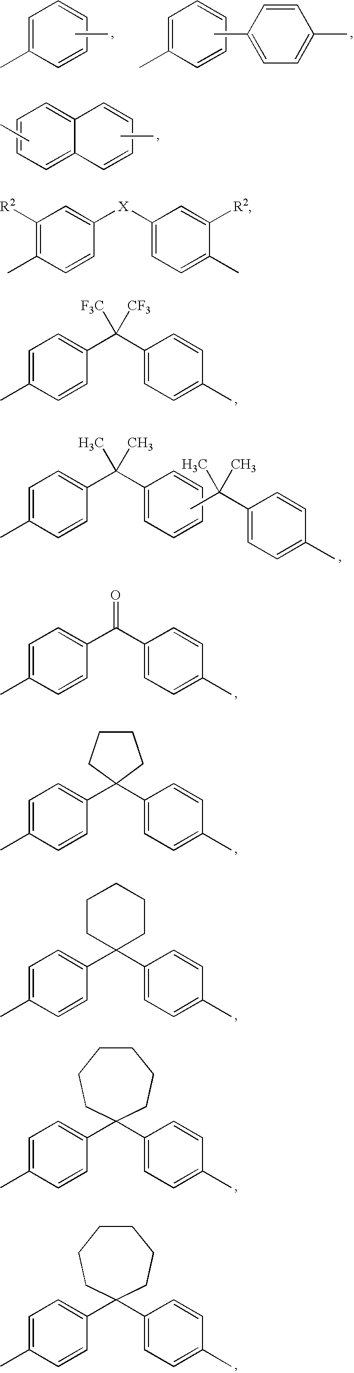 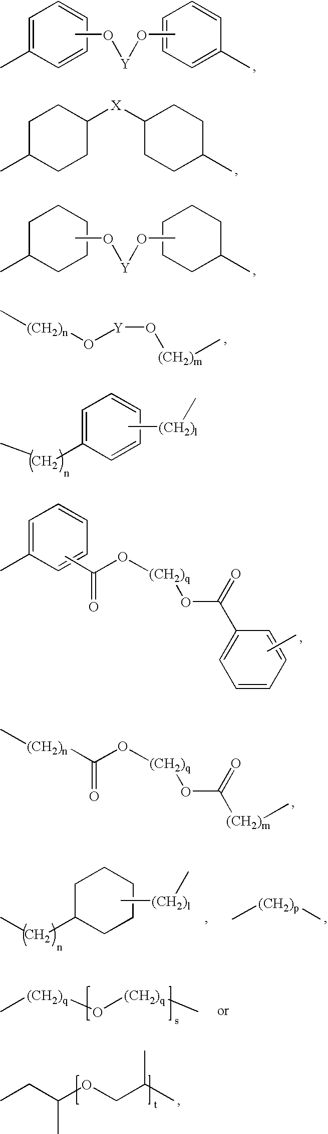
B² is a bridge of the formula 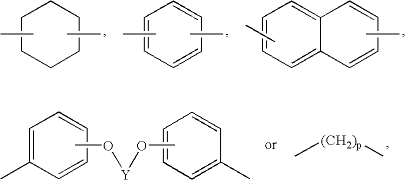
R² is hydrogen, methyl, ethyl, methoxy or fluorine,
X is a bridge of the formula 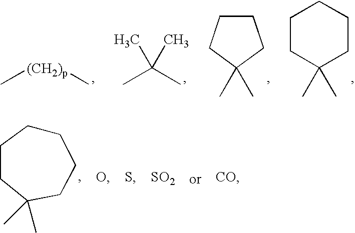
Y is a bridge of the formula 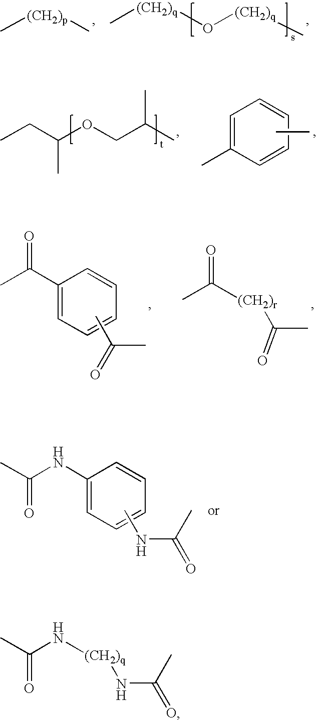
l is an integer from 0 to 8,
m, n, p and r independently of one another are an integer from 1 to 8,
q is an integer from 2 to 8 and
s and t independently of one another are an integer from 1 to 4,
R²⁰ and R²² independently of one another are cyano or optionally substituted C₁-C₈-alkoxycarbonyl,
R²¹ is hydrogen or optionally substituted C₁-C₃-alkyl
R²³ is hydrogen, unsubstituted or substituted C₁-C₈-alkyl or unsubstituted or substituted C₁-C₈-alkoxycarbonyl,
R²⁴ is cyano, unsubstituted or substituted C₁-C₈-alkylsulphonyl or unsubstituted or substituted C₁-C₈-alkoxycarbonyl,
R²⁵ is unsubstituted or substituted C₁-C₈-alkyl, unsubstituted or substituted C₃-C₇-cycloalkyl, unsubstituted or substituted C₆-C₁₀-aryl,
R²⁶ is hydrogen or independently of R²⁵ has the definition of R²⁵, or
NR²⁵R²⁶ is pyrrolidino, piperidino or morpholino,
R²⁷ is one or more radicals having the definition hydrogen, unsubstituted or substituted C₁-C₈-alkyl, unsubstituted or substituted C₁-C₈-alkoxy, fluorine, cyano, unsubstituted or substituted C₃-C₇-cycloalkyl or unsubstituted or substituted C₆-C₁₀-aryl,
R²⁸ is hydrogen or unsubstituted or substituted C₁-C₈-alkyl,
and at least one orange dye of the formula (L), (LI), (LII), (LIII), (LIV) or (LV) 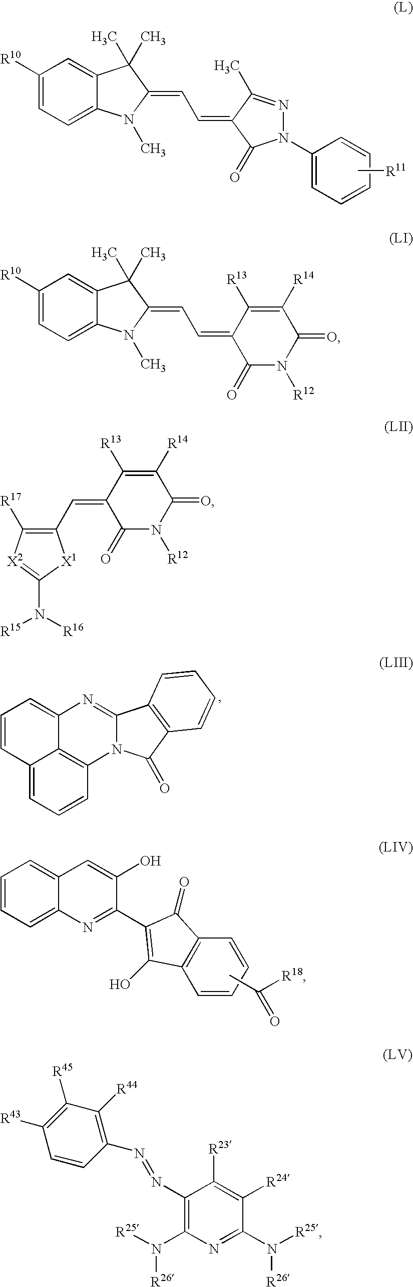
in which
R¹⁰ is hydrogen, unsubstituted or substituted C₁-C₈-alkyl, unsubstituted or substituted C₁-C₈-alkoxy, unsubstituted or substituted C₁-C₈-alkylsulphonyl unsubstituted or substituted C₁-C₈-alkoxycarbonyl nitro, cyano or fluorine,
R¹¹ is hydrogen, unsubstituted or substituted C₁-C₈-alkyl, unsubstituted or substituted C₁-C₈alkoxy, optionally substituted C₁-C₈-alkoxycarbonyl, nitro, cyano or fluorine,
R¹² is hydrogen, unsubstituted or substituted C₁-C₈-alkyl or unsubstituted or substituted C₃-C₈-cycloalkyl,
R¹³ is hydrogen, unsubstituted or substituted C₁-C₈-alkyl or unsubstituted or substituted C₁-C₈-alkoxycarbonyl,
R¹⁴ is cyano, unsubstituted or substituted C₁-C₈-alkylsulphonyl or unsubstituted or substituted C₁-C₈-alkoxycarbonyl,
X¹ is O or S,
X² is N or C—CN,
R¹⁵ and R¹⁶ independently of one another are unsubstituted or substituted C₁-C₈-alkyl, unsubstituted or substituted C₃-C₈-cycloalkyl or unsubstituted or y substituted C₆-C₁₀-aryl, or
NR¹⁵R¹⁶ is pyrrolidino, piperidino or morpholino,
R¹⁷ is hydrogen or C₁-C₈-alkyl,
R¹⁸ is unsubstituted or substituted C₆-C₁₀-aryl,
R²³′ is hydrogen, unsubstituted or substituted C₁-C₈-alkyl or unsubstituted or substituted C₁-C₈-alkoxycarbonyl,
R²⁴′ is cyano, unsubstituted or substituted C₁-C₈-alkylsulphonyl or unsubstituted or substituted C₁-C₈-alkoxycarbonyl,
R²⁵′ is unsubstituted or substituted C₁-C₈-alkyl, optionally substituted C₃-C₇-cycloalkyl, unsubstituted or substituted C₆-C₁₀-aryl,
R^{26′} is hydrogen or independently of R²⁵′ has the definition of R²⁵′, or
NR²⁵′R²⁶′ is pyrrolidino, piperidino or morpholino,
R⁴³ and R⁴⁴ independently of one another are hydrogen, unsubstituted or substituted C₁-C₄-alkyl, nitro, cyano, C₁-C₄-alkoxycarbonyl, and
R⁴⁵ is hydrogen, methyl, fluorine or chlorine.

2. The mixture according to claim 1, comprising a red dye of the formula (I) and at least one orange dye of the formula (L), (LI), (LII), (LIII), (LIV) or (LV).

3. The mixture according to claim 1, comprising a red dye of the formula (I) and at least one orange dye of the formula (L).

4. The mixture according to claim 1, comprising a red dye of the formula (C) and at least one orange dye of the formula (L), (LI), (LII), (LIII), (LIV) or (LV), (L).

5. The mixture according to claim 1, comprising a red dye of the formula (C) and at least one orange dye of the formula (L).

6. The mixtures according to claim 1, comprising a red dye of the formula (CI) and at least one orange dye of the formula (L), (LI), (LII), (LIII), (LIV) or (LV).

7. The mixtures according to claim 1, comprising a red dye of the formula (CI) and at least one orange dye of the formula (L).

8. Mixture according to claim 1, comprising a red dye of the formula I
wherein
R¹ is hydrogen or methyl,
B¹ is a bridge of the formula 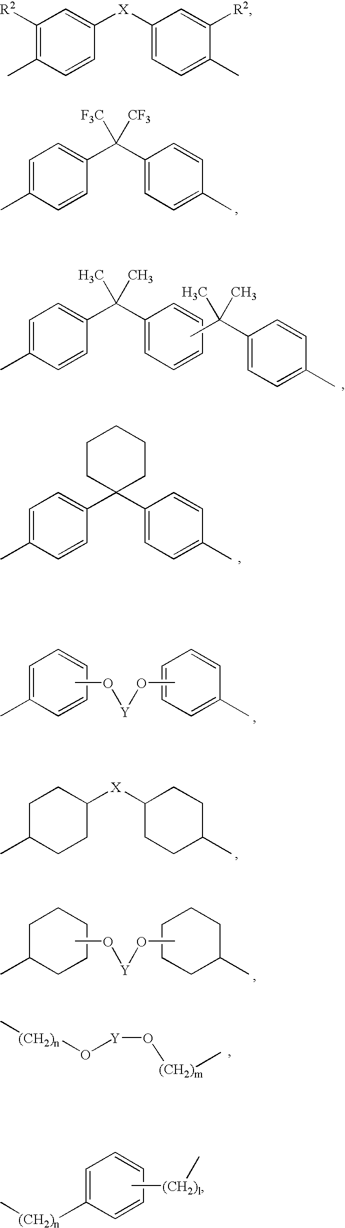 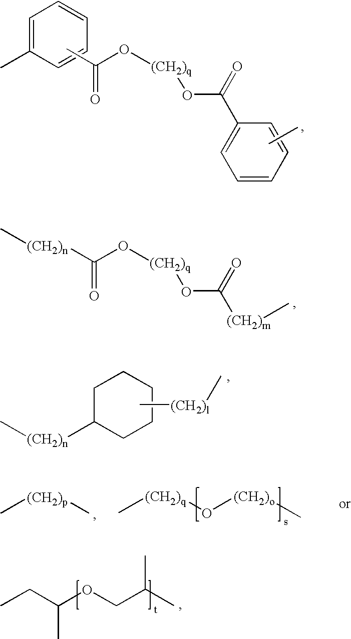
R² is methyl or chlorine,
X is a bridge of the formula 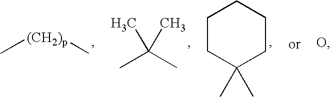
Y is a bridge of the formula 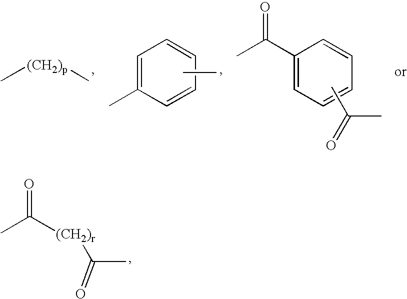
l is an integer from 0 to 4,
m, n and p independently of one another are an integer from 1 to 4,
q and r independently of one another are an integer from 2 to 6,
s and t independently of one another are an integer from 1 to 2 and
the undefined ring positions are 1,2-, 1,3- or 1,4-.

9. The mixture according to claim 1, comprising a red dye of the formula (I),
wherein
R¹ is hydrogen or methyl,
B² is a bridge of the formula 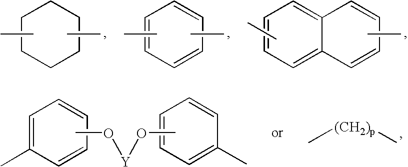
Y is a bridge of the formula 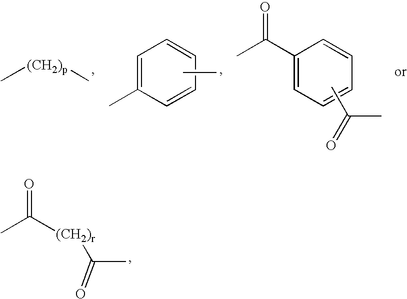
p is an integer from 1 to 6, and
r is an integer from 2 to 6 and
the undefined ring positions in the case of benzene or cyclohexane rings are 1,2-, 1,3- or 1,4- and in the case of the naphthalene ring are 2,6- and 2,7-.

10. The mixture according to claim 1, comprising a red dye of the formula (C),
wherein
R²⁰ is cyano, methoxycarbonyl or ethoxycarbonyl,
R²² is cyano,
R²¹ is hydrogen or methyl,
R²³ is methyl or methoxycarbonyl,
R²⁴ is cyano, methanesulphonyl, methoxycarbonyl or ethoxycarbonyl,
R²⁵ is methyl, propyl, butyl, methoxypropyl, cyanoethyl, benzyl, cyclohexyl, phenyl or tolyl more preferably ethyl, propyl, butyl, methoxyethyl, methoxypropyl, benzyl, cyclohexyl or phenyl,
R²⁶ is hydrogen or independently of R²⁵ has the definition of R²⁵, or
NR²⁵R²⁶ is pyrrolidino, piperidino or morpholino,
and an orange dye of the formula (L).

11. The mixture according to claim 1, comprising a red dye of the formula (C) wherein
R²⁰ is COOCH₃, R²¹ and R²³ is CH₃, R²² and R²⁴ are CN, R²⁵ is —CH₂CH₂CH₂OCH₃ and R¹⁶ is H
and an orange dye of the formula (L) wherein
R¹⁰ is COOCH₃ and R¹¹ is H.

12. The mixture according to claim 1 further comprising at least
a) a yellow dye and at least one green dye or
b) a yellow dye and at least one blue dye.

13. The mixture according to claim 1, further comprising at least
a) a yellow dye, with a λₘₐₓ of 420 to 460 nm and at least one green dye, with a λₘₐₓ of 610 to 700 nm, or
b) a yellow dye, with a λₘₐₓ of 420 to 460 nm, and at least one blue dye, with a λₘₐₓ of 570 to 640 nm.

14. The mixtures according to claim 1, further comprising
a) at least one yellow dye of the formula (CCI), (CCII), (CCIII), (CCIV) or (CCV) 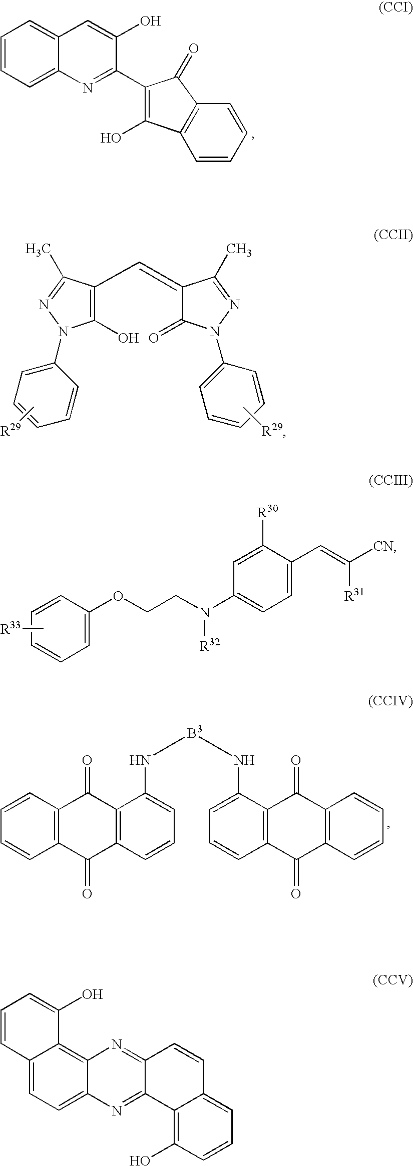
wherein
R²⁹ is one or more radicals having the definition hydrogen, unsubstituted or substituted C₁-C₈-alkyl, unsubstituted or substituted C₁-C₈-alkoxy, fluorine, cyano, unsubstituted or substituted C₃-C₇-cycloalkyl or unsubstituted or substituted C₆-C₁₀-aryl
R³⁰ is hydrogen, methyl, ethyl or methoxy,
R³¹ is cyano, C₁-C₈-alkoxycarbonyl or C₁-C₈-alkanesulphonyl,
R³² is hydrogen, unsubstituted or substituted C₁-C₈-alkyl or unsubstituted or substituted C₃-C₇-cycloalkyl,
R³³ is hydrogen, unsubstituted or substituted C₁-C₈-alkyl, unsubstituted or substituted C₁-C₈-alkoxy, optionally substituted C₃-C₇-cycloalkyl or unsubstituted or substituted C₆-C₁₀-aryl,
and
B³ is a bridge of the formula 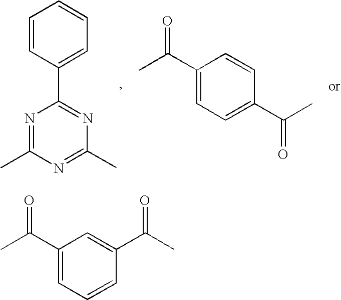
and at least one green dye of the formula (CCLI) 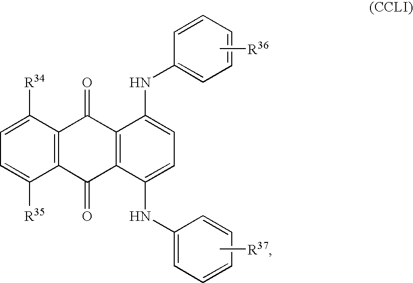
wherein
R³⁴ and R³⁵ independently of one another are hydrogen, hydroxyl or C₁-C₄-alkoxy,
R³⁶ and R³⁷ independently of one another are hydrogen, unsubstituted or substituted C₁-C₈-alkyl, unsubstituted or substituted C₁-C₈-alkoxy, fluorine, unsubstituted or substituted C₃-C₇-cycloalkyl or unsubstituted or substituted C₆-C₁₀-aryl,
or
b) at least one yellow dye of the formula (CCI), (CCII), (CCIII), (CCIV) or (CCV) and at least one blue dye of the formula (CCCI) 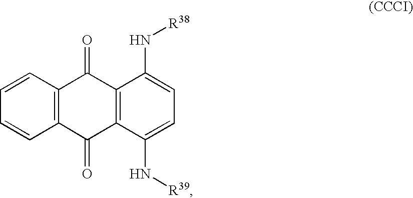
wherein
R³⁸ and R³⁹ independently of one another are unsubstituted or substituted C₁-C₈-alkyl, unsubstituted or substituted C₃-C₇-cycloalkyl or unsubstituted or substituted C₆-C₁₀-aryl.

15. The masterbatch containing 15%-80% by weight of a mixture according to claim 1 and a carrier.

16. A method for the mass colouring of plastics wherein a mixture according to claim 1 is applied.

17. A method of mass-colouring plastics, wherein the mixture according to claim 1 is incorporated into the melted mass of plastic or actually added to the starting components for producing the plastic prior to the polymerization.

18. A plastic comprising a mixture according to claim 1.

19. A compound of the formula I 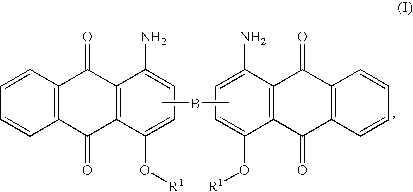
in which
—B— is —O—B¹—O— or —CH₂—B²—CH₂—,
R¹ is hydrogen, optionally substituted C₁-C₈-alkyl or unsubstituted or substituted C₃-C₈-cycloalkyl,
B¹ is a bridge of the formula 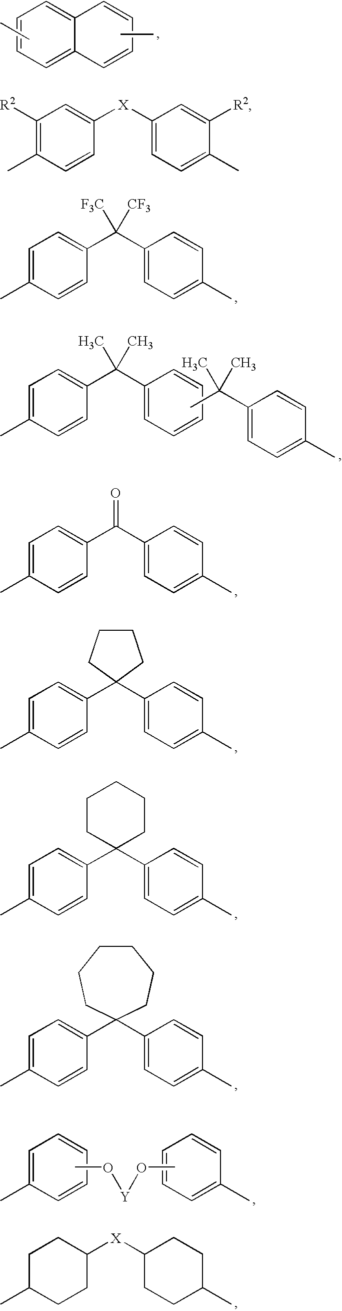 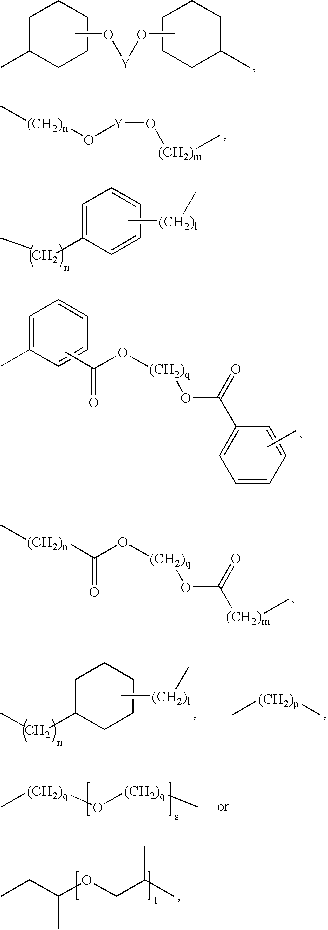
B² is a bridge of the formula 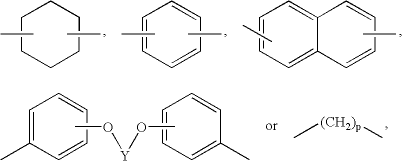
R² is methyl, ethyl methoxy or fluorine,
X is a bridge of the formula 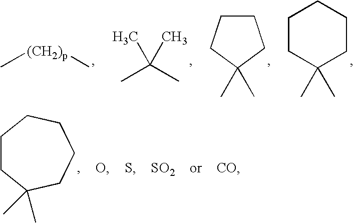
Y is a bridge of the formula 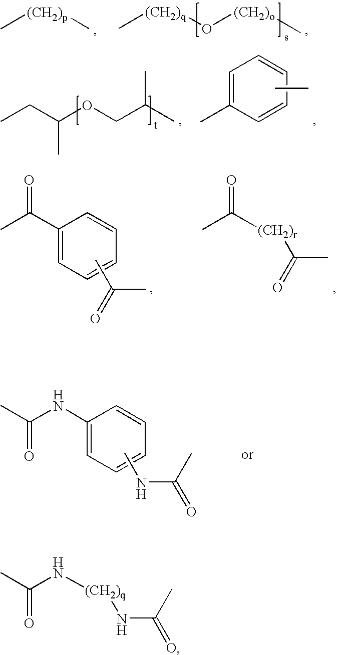
l is an integer from 0 to 8,
m, n, p and r independently of one another are an integer from 1 to 8,
q is an integer from 2 to 8 and
s and t independently of one another are an integer from 1 to 4.

20. The compound of the formula 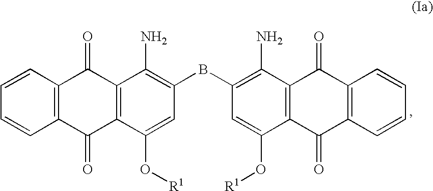
wherein
—B— is —O—B¹—O— or —CH₂—B²—CH₂—,
R¹ is hydrogen, optionally substituted C₁-C₈-alkyl or unsubstituted or substituted C₃-C₈-cycloalkyl,
B¹ is a bridge of the formula 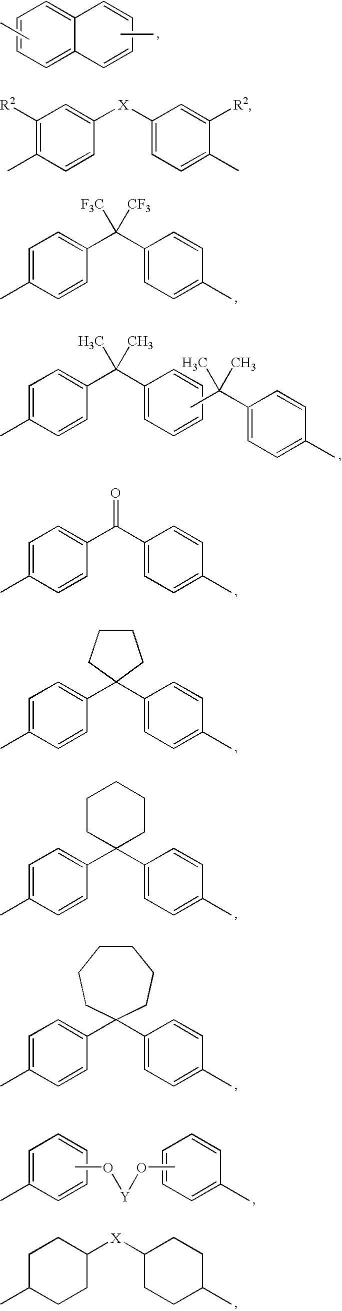 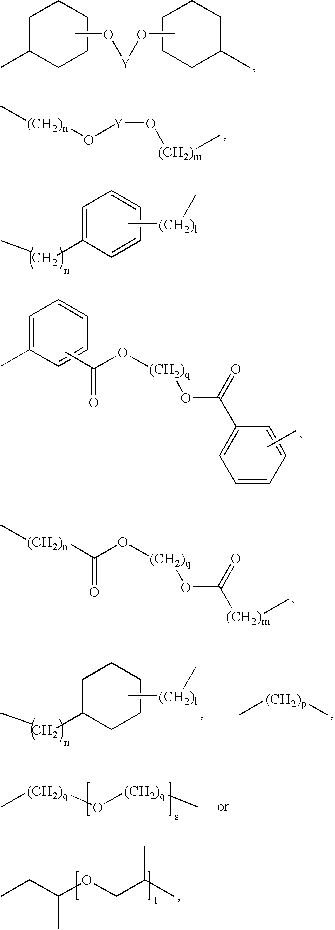
B² is a bridge of the formula 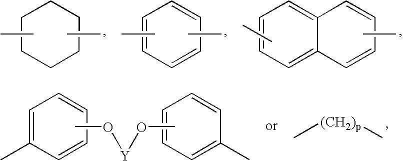
R² is methyl, ethyl, methoxy or fluorine,
X is a bridge of the formula 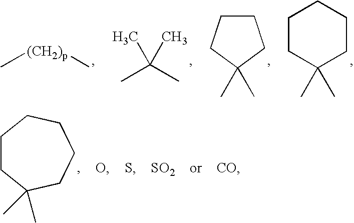
Y is a bridge of the formula 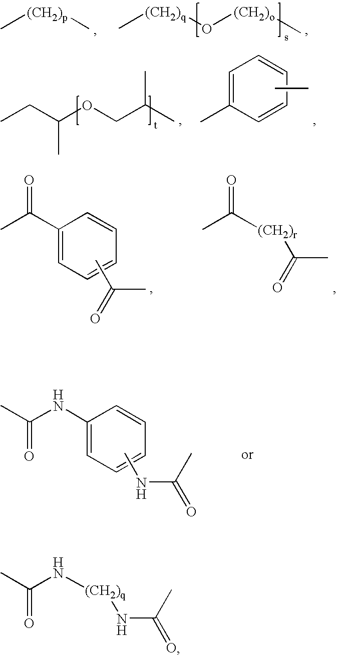
l is an integer from 0 to 8,
m, n, p and r independently of one another are an integer from 1 to 8,
q is an integer from 2 to 8 and
s and t independently of one another are an integer from 1 to 4.

21. The compound according to claim 19, wherein
R¹ is hydrogen or methyl,
B¹ is a bridge of the formula 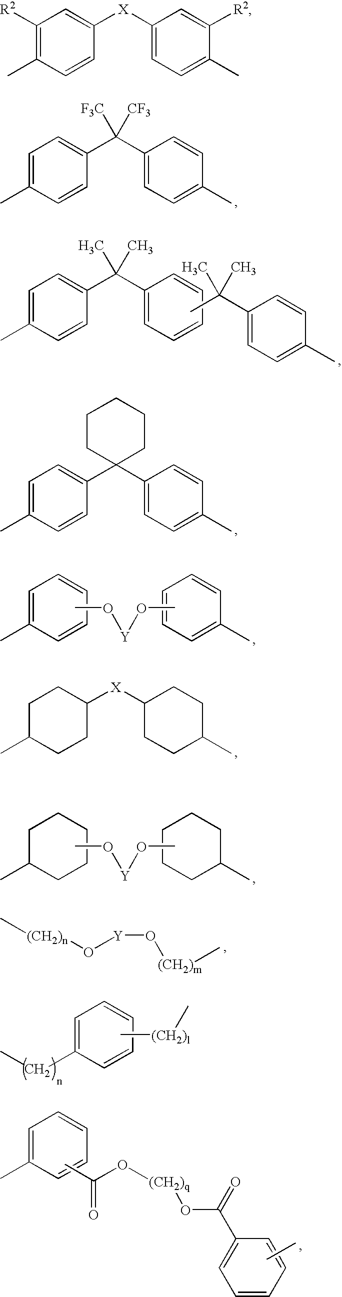 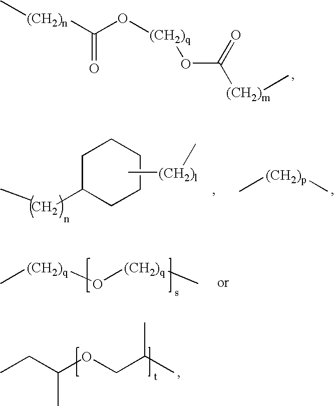
R² is methyl,
X is a bridge of the formula 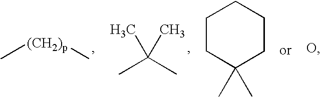
Y is a bridge of the formula 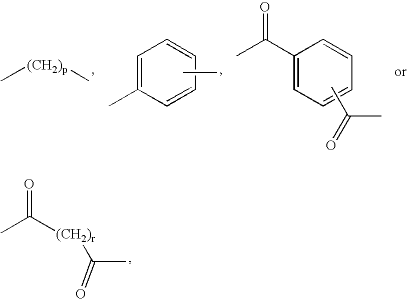
l is an integer from 0 to 4,
m, n and p independently of one another are an integer from 1 to 4,
q and r independently of one another are an integer from 2 to 6,
s and t independently of one another are an integer from 1 to 2 and
the undefined ring positions are 1,2-, 1,3- or 1,4-.

22. The compound according to claim 19, wherein
R¹ is hydrogen or methyl,
B² is a bridge of the formula 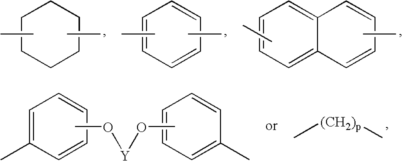
Y is a bridge of the formula 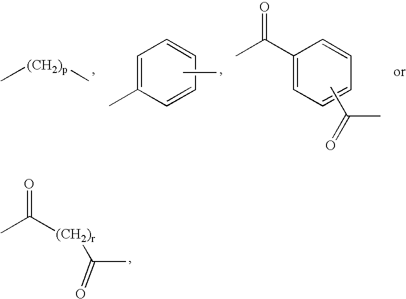
p is an integer from 1 to 6, and
r is an integer from 2 to 6 and
the undefined ring positions in the case of benzene or cyclohexane rings are 1,2-, 1,3- or 1,4- and in the case of the naphthalene ring are 2,6- and 2,7-.

23. A process for preparing the dyes of the compound of the formula (I)
in which
B is a bridge —O—B¹—O—, according to claim 19,
wherein an anthraquinone compound of the formula (II) 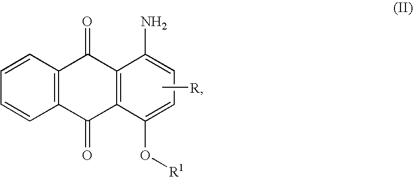
in which
R¹ is hydrogen, unsubstituted or substituted C₁-C₈-alkyl or unsubstituted or substituted C₃-C₈-cycloalkyl and
R is chlorine, bromine or phenoxy,
is reacted with a bifunctional alcohol of the formula (III)
HO—B¹—OH (III),
in which
B¹ is a bridge of the formula 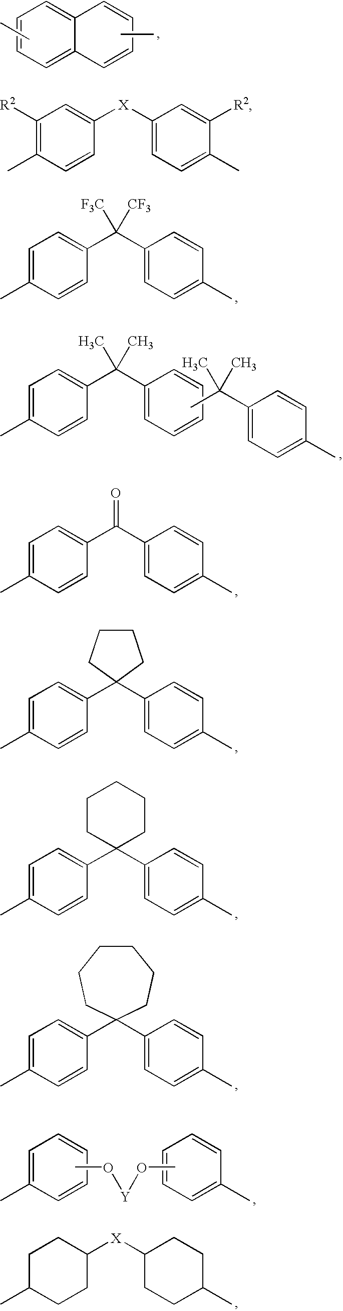 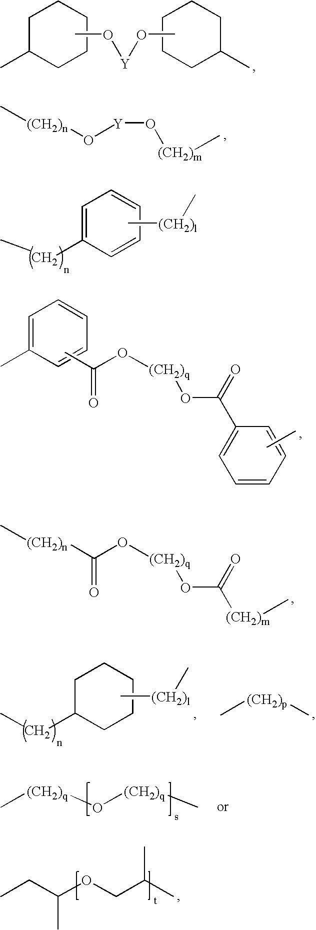
R² is methyl, ethyl, methoxy or fluorine,
X is a bridge of the formula 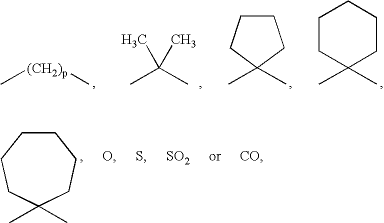
Y is a bridge of the formula 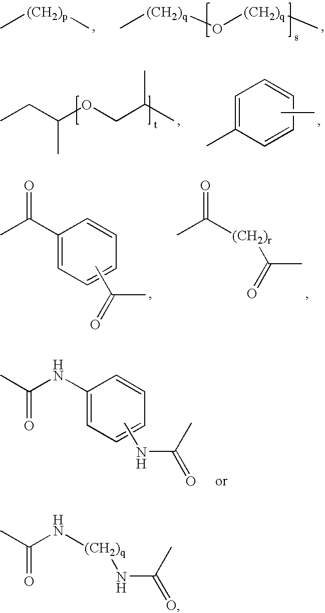
l is an integer from 0 to 8.
m, n, p and r independently of one another are an integer from 1 to 8,
q is an integer from 2 to 8 and
and t independently of one another are an integer from 1 to 4.

24. A process for preparing compounds of the formula (I) according to claim 19 in which
B is a bridge —O—B¹—O— and
B¹ is a bridge of the formula 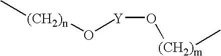
in which
Y is a bifunctional group of the formula 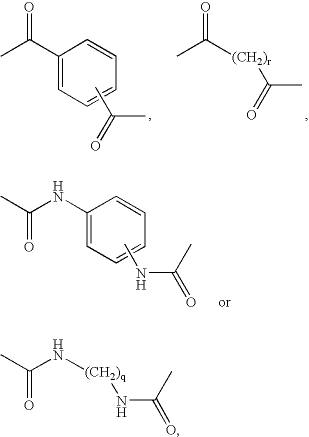
m, n and r independently of one another are an integer from 1 to 8 and n and m are alike and
q is an integer from 2 to 8,
wherein an anthraquinone compound of the formula (IV) 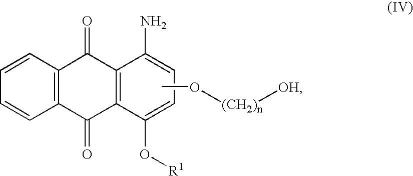
in which
R¹ is hydrogen, unsubstituted or substituted C₁-C₈-alkyl or unsubstituted or substituted C₃-C₈-cycloalkyl and
n is an integer from 1 to 8,
is reacted with a bifunctional acid chloride or ester of the formulae (V) 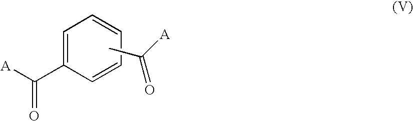
or (VI)
A-CO—(CH₂)ᵣ—CO-A (VI)
or with a bifunctional isocyanate of the formula (VII) 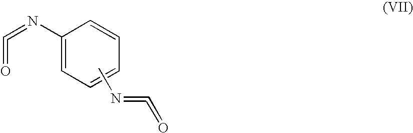
or (VIII)
O═C═N—(CH₂)_{q}—N═C═O (VIII)
in which
A is Cl or methoxy,
r is an integer from 1 to 8 and
q is an integer from 2 to 8.

25. A process for preparing the dyes of the formula (I) according to claim 19 in which
B is a bridge —O—B¹—O—,
and
B¹ is a bridge of the formula 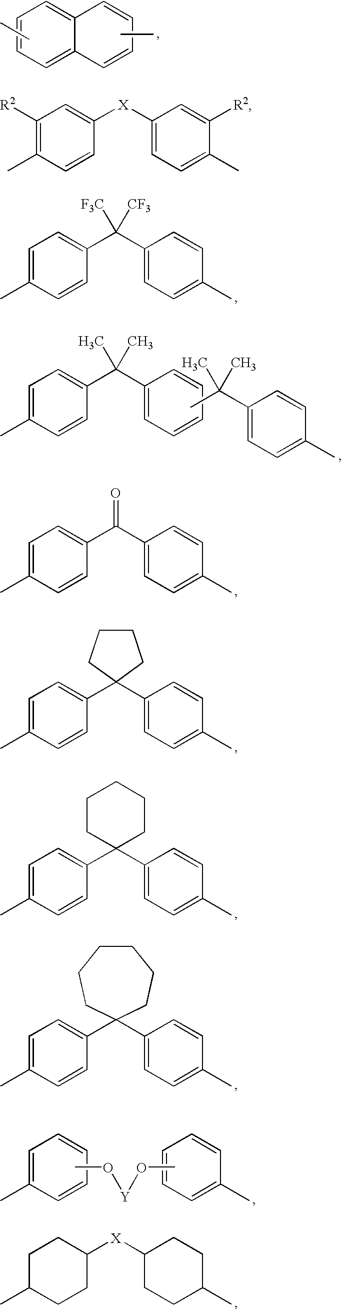 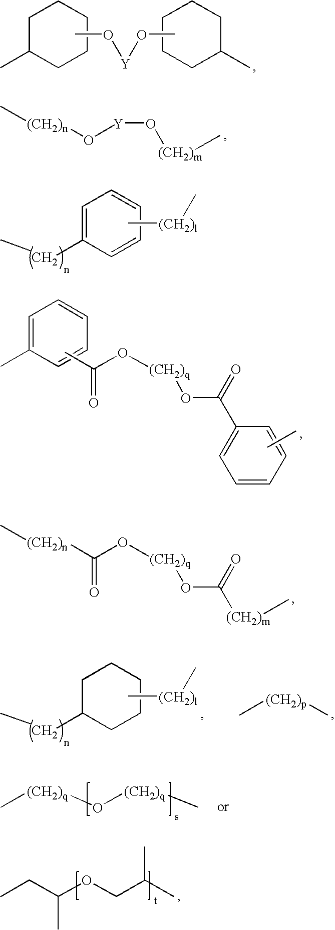
R² is methyl, ethyl, methoxy or fluorine,
X is a bridge of the formula 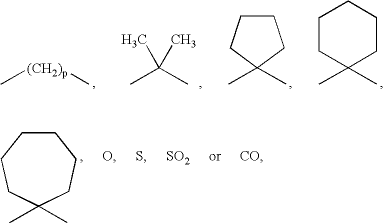
Y is a bridge of the formula 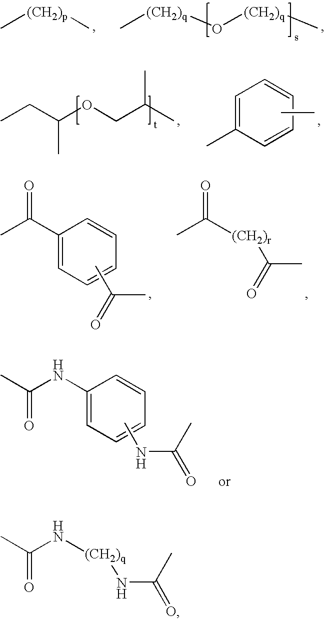
l is an integer from 0 to 8,
m, n, p and r independently of one another are an integer from 1 to 8,
q is an integer from 2 to 8 and
s and t independently of one another are an integer from 1 to 4,
wherein an anthraquinone compound of the formula (II) 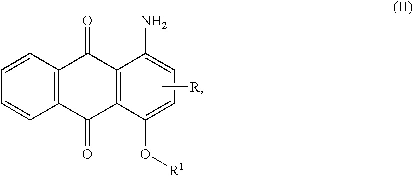
in which
R¹ is hydrogen, unsubstituted or substituted C₁-C₈-alkyl or unsubstituted or substituted C₃-C₈-cycloalkyl and
R is chlorine, bromine or phenoxy,
is reacted with an anthraquinone compound of the formula (IX) 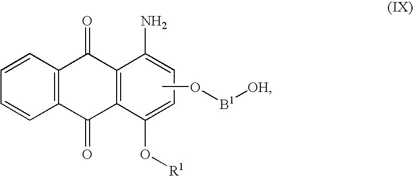
in which
R¹ is hydrogen, unsubstituted or substituted C₁-C₈-alkyl or unsubstituted or substituted C₁-C₈-cycloalkyl.

26. Process for preparing the dyes of the formula (I) according to claim 19 in which
B is a bridge —CH₂—B²—CH₂—,
wherein a dihydroanthraquinone compound which in one tautomeric form corresponds to the formula (X) 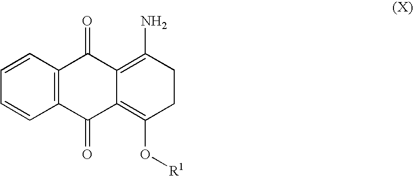
in which
R¹ is hydrogen, unsubstituted or substituted C₁ to C₈-alkyl or unsubstituted or substituted C₃ to C₈-cycloalkyl,
is reacted with a bifunctional aldehyde of the formula (XI)
OCH—B²—CHO (XI),
in which
B² is a bridge of the formulae 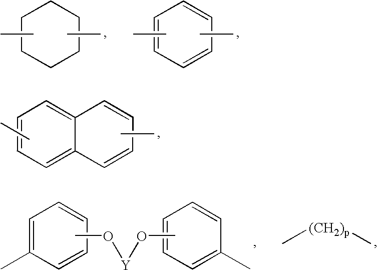
Y is a bridge of the formulae 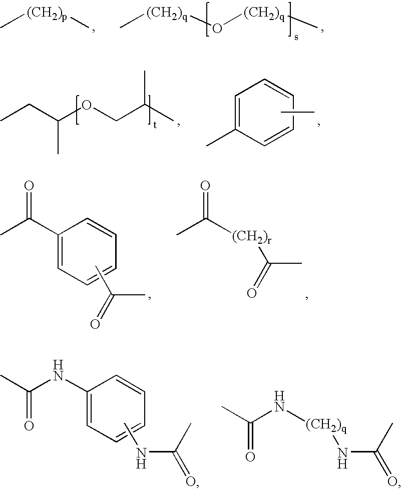
p and r independently of one another are an integer from 1 to 8,
q is an integer from 2 to 8 and
s and t independently of one another are an integer from to 4.

27. A masterbatch containing 15%-90%, by weight of a compound of the formula I according to claim 19 and a carrier.

28. A method for the mass colouring of plastics, wherein a compound according to claim 19 is applied.

29. A method of mass-colouring plastics, characterized in that a compound according to claim 19 is incorporated into the melted plastic mass or actually added to the starting components for producing the plastic, prior to the polymerization.

30. A plastic comprising a compound according to claim 19.

31. A method for colouring synthetic fibres, preferably in dispersed form, wherein a mixture according to claim 1 or a compound according to claim 19 is applied.
